SEQUENCE LISTING (11 sequences)

SEQ ID NO: 1 - DNA, 24 bp, Artificial
  Probe
gccagacaaacactttagccatta

SEQ ID NO: 2 - DNA, 26 bp, Artificial
  Probe
tacctgtggtcctcatttgtagctat

SEQ ID NO: 3 - DNA, 29 bp, Artificial
  Probe
ctggacacattccgtaatatcccacctcc

SEQ ID NO: 4 - DNA, 58 bp, Artificial
  Primer
cttttctttttctttttctcttttctttttctttttcccttactacattcaaggggat

SEQ ID NO: 5 - DNA, 56 bp, Artificial
  Primer
cttccctgttctgccaatctgcactggatgttcagagacgtctgttggtggtctcc

SEQ ID NO: 6 - DNA, 44 bp, Artificial
  Primer
tggggatgagagaaagaggaggacgcaaaaagccactaccactg

SEQ ID NO: 7 - DNA, 41 bp, Artificial
  Primer
caggtttgagccatcatgcatccaatccctaagcttgactg

SEQ ID NO: 8 - DNA, 47 bp, Artificial
  Primer
gatgattgtctttttcctcttgcctgagctatcttaagaaatacatg

SEQ ID NO: 9 - DNA, 41 bp, Artificial
  Primer
agtaaatgctgcagttcagaggccgtggcatatcatccccc

SEQ ID NO: 10 - DNA, 43 bp, Artificial
  Primer
cccagactgcttcaaaattaccgagcctcatctgtacttctgc

SEQ ID NO: 11 - DNA, 47 bp, Artificial
  Primer
gcattaagagtaaaattcctcttacatgaccaccaggtaggtgtatt

(19) United States
(12) Patent Application Publication
(10) Pub. No.: US 2008/0003592 A1
(43) Pub. Date: Jan. 3, 2008

(54) Method for conducting pharmacogenomics-based studies

(76) Inventor: James L. Novakoff, Boca Raton, FL (US)

Correspondence Address: NIXON PEABODY, LLP, 401 9TH STREET, NW, SUITE 900, WASHINGTON, DC, 20004-2128, US

(21) Appl. No.: 11/600,341

(22) Filed: Nov. 15, 2006

Related U.S. Application Data
(63) Continuation of application No. 11/013,533, filed on Dec. 17, 2004, now abandoned.
(60) Provisional application No. 60/531,430, filed on Dec. 19, 2003.

(51) Int. Cl. C12Q 1/68

(52) U.S. Cl. 435/6

(57) ABSTRACT

The present invention relates to processes and methods for undertaking clinical trials using pharmacogenomics-based techniques. In particular, the present invention relates to the collection of circulating RNA in an individual or group of individuals before and after an event or intervention, identifying any changes in circulating RNA before and after such event or intervention and relating such change to the event or intervention but without the need for identification of the protein for which such RNA codes; and using changes in the levels of these RNA transcripts to assess disease progression, remission, therapeutic effect, or development of new treatments.

This application is a continuation, and claims the benefit under 35 U.S.C. § 120, of U.S. application Ser. No. 11/013,533, filed on Dec. 17, 2004, which claims the benefit under 35 U.S.C. § 119(e) of U.S. provisional application Ser. No. 60/531,430, filed on Dec. 19, 2003, entitled “Method For Conducting Pharmacogenomics-Based Studies,” which are incorporated herein by reference.

### BACKGROUND OF THE INVENTION

This invention is in the field of gene expression, including methods for high and low density microarray gene expression profiling, as well as the profiling individual genes or small collections of genes. This application concerns the effects of medical events and interventions on gene expression in an individual or group of individuals.

Only a fraction of the total number of genes present in the genome is expressed in any given cell. The total number of genes that are expressed in a cell determine its properties, including development and differentiation, homeostasis, its response to insults, cell cycle regulation, aging, apoptosis, and the like. Alterations in gene expression can determine the course of normal cell development and the appearance of diseased states, such as cancer, or can occur in response to stimuli. Because the profile of gene expression in cells has direct consequences, methods for analyzing gene expression are important. Identification of gene-expression profiles will not only further an understanding of normal biological processes in subjects but provides key information relevant to prognosis and treatment of a variety of diseases or conditions in humans having alterations in gene expression. In addition, since differential gene expression is associated with predisposition to certain diseases, infectious agents and responsiveness to external treatments, identification of such gene-expression profiles can provide a powerful diagnostic or prognostic tool for diseases, and as a tool to identify new drugs or monitor the use of drugs for treating or preventing such diseases.

The profiles of gene expression in any given cell directly reflect the properties and functions of the cell. A large scale analysis of the global expression pattern during development and in the adult in different tissues and cells provides expression profiles of all genes expressed in that cell/tissue. Such gene expression profiles provide important information on gene function and normal biological processes in organisms.

Disease states and progression of disease may be dictated by the altered expression of certain genes, and gene expression profiling provides a powerful tool to characterize the disease state and clinical consequences, responsiveness to different drugs, and predicted disease outcome. A reliable gene expression profiling technique would provide the means to rapidly identify the critical genes that indicate a subject's response to disease states, drug treatment, and course of therapy. Such information may thereafter be used for diagnosis using a smaller scale analysis using for instance the real-time polymerase chain reaction (PCR) and examining a small collection of genes.

### SUMMARY OF THE INVENTION

The present invention provides methods for analyzing gene expression of a subject in a variety of settings that may be before, during or after a medical event including, but not limited to, treatment with an approved drug, treatment with an experimental drug during an clinical trial, trauma, surgery, preventative therapy, vaccination, drug dosing determination, drug efficacy determination, progress or course of therapy with a drug, monitoring disease stage or status or progression, aging, drug addiction, weight loss or gain, cardiovascular or other cardiac-related events, reactions to treatment with a drug, exposure to radiation or other environmental event, exposure to weightlessness or other environmental conditions, exposure to chemical or biological agents (both natural and man-made), diet (ingestion of foodstuffs). In addition, the present invention provides a database of gene expression data for a subject or group of subjects obtained before, during or after a medical event. In one embodiment, the gene expression data obtained according to the present invention is from a subject involved in a clinical trial. In another embodiment, the gene expression data identifies any gene, or collection of genes, that undergoes a change in its level of expression without regard for the function of the encoded protein or association of the gene with any particular function, pathway, disease or other attribute other than its ability to be detected.

In another embodiment, the gene or genes of interest may be known to have an association with the gene expression profile of the subject or the medical event of interest. In one embodiment, for example, a gene known to predispose a subject to a particular tumor formation when expressed, may be monitored before any symptoms are present in the subject to establish a baseline expression level in that subject. Monitoring the gene expression level changes in the patient may identify early tumor formation and an opportunity to treat, suppress or prevent disease, cardiac conditions, psychological conditions including depression and anxiety, Alzheimer's, arthritic and other chronic and non-chronic diseases as detailed in The Merck Manual of Diagnosis and Therapy (Beers & Berkow, Eds.).

The methods for analyzing gene expression include obtaining a nucleic acid sample from a subject, such a RNA. The target sequences are obtained in any of a number of manners, such as by performing reverse transcription on a set of mRNA molecules and amplification. The mRNA molecules are optionally derived from a patient's tissues, organs, cells, organisms, or cell cultures, which have been or are to be exposed to one or more specific treatments that potentially alter the biological state of the cell, organism, or cell culture.

The one or more RNA members are detected by any of a number of techniques, thereby generating one or more sets of gene expression data. Detection is performed, for example, by measuring the presence, absence, or quantity/amplitude of one or more properties of the expressed genes. Example properties of the amplification products include, but are not limited to, mass, light absorption or emission, and one or more electrochemical properties. One or more expressed genes are detected and the information collected is used to generate a set of gene expression data.

The set of gene expression data may be stored in a database. This data is then used for a variety of analyses including, but not limited to, performing a comparative analysis (for example, by measuring a ratio of each target gene to each reference gene or other analysis of interest).

The present invention also provides methods for analyzing gene expression including the steps of obtaining RNA or cDNA from a plurality of samples for a plurality of target sequences; quantifying the levels of individual expressed genes, thereby generating a set of gene expression data; storing the set of gene expression data in a database; and performing a comparative analysis of the set of gene expression data.

The optional amplification reaction used in the methods of the present invention includes, but is not limited to, a polymerase chain reaction, a transcription-based amplification, a self-sustained sequence replication, a nucleic acid sequence based amplification, a ligase chain reaction, a ligase detection reaction, a strand displacement amplification, a repair chain reaction, a cyclic probe reaction, a rapid amplification of cDNA ends, an invader assay, a bridge amplification or rolling circle amplification, or a combination thereof.

The present invention also provides methods for analyzing gene expression including the steps of obtaining RNA or cDNA from multiple samples; and detecting and quantifying the expressed gene products using a high throughput platform, wherein detecting and quantifying generates a set of gene expression data; storing the set of gene expression data in a database; and performing a comparative analysis of the set of gene expression data.

The methods of the present invention optionally include performing one or more of the amplifying, separating or detecting steps in a high throughput format. For example, the reactions can be performed in multi-well plates. Optionally, anywhere between about one and about 5000 reactions, between about 50 and 2000 reactions, and about 100 reactions, are performed per hour using the methods of the present invention. Furthermore, one or more miniaturized scale platforms can be used to perform the methods of the present invention.

The present invention may also utilize systems for analyzing gene expression. The elements of the system include, but are not limited to, a) an amplification module for producing a plurality of amplification products from a pool of target sequences; b) a detection module for detecting one or more members of the plurality of amplification products and generating a set of gene expression data comprising a plurality of data points; and c) an analyzing module in operational communication with the detection module, the analyzing module comprising a computer or computer-readable medium comprising one or more logical instructions which organize the plurality of data points into a database and one or more logical instructions which analyze the plurality of data points. Any or all of these modules can comprise high throughput technologies and/or systems.

The amplification module of the present invention includes at least one pair of universal primers and at least one pair of target-specific primers for use in the amplification process. Optionally, the amplification module includes a unique pair of universal primers for each target sequence. Furthermore, the amplification module can include components to perform one or more of the following reactions: a polymerase chain reaction, a transcription-based amplification, a self-sustained sequence replication, a nucleic acid sequence based amplification, a ligase chain reaction, a ligase detection reaction, a strand displacement amplification, a repair chain reaction, a cyclic probe reaction, a rapid amplification of cDNA ends, an invader assay, or various solution phase and/or solid phase assays (for example, bridge amplification or rolling circle amplification). The detection module can include systems for implementing separation of the amplification products; exemplary detection modules include, but are not limited to, mass spectrometry instrumentation and electrophoretic devices.

The analyzing module of the system includes one or more logical instructions for analyzing the plurality of data points generated by the detection system. For example, the instructions can include software for performing difference analysis upon the plurality of data points. Additionally (or alternatively), the instructions can include or be embodied in software for generating a graphical representation of the plurality of data points. Optionally, the instructions can be embodied in system software which performs combinatorial analysis on the plurality of data points.

The present invention also provides kits for obtaining a set of amplification products of target genes and reference-gene to generate the gene expression profiles. The kits of the present invention include a) at least one pair of universal primers; b) at least one pair of target-specific primers; c) at least one pair of reference gene-specific primers; and d) one or more amplification reaction enzymes, reagents, or buffers. The kits optionally further include software for storing and analyzing data obtained from the amplification reactions.

Additionally, the present invention provides compositions for preparing a plurality of amplification products from a plurality of mRNA target sequences. The compositions include one or more pairs of universal primers; and one or more pairs of target-specific primers. The present invention also provides for the use of the kits of the present invention for practicing any of the methods of the present invention, as well as the use of a composition or kit as provided by the present invention for practicing a method of the present invention. Furthermore, the present invention provides assays utilizing any of these uses.

### BRIEF DESCRIPTION OF THE DRAWINGS

FIG. 1: Shows a graphical data plot of the gene-specific analysis.

### DETAILED DESCRIPTION OF THE INVENTION

Drugs are often identified in high throughput screens by selection of a single or a few properties. Thus, a primary molecular target is identified but the full pathway as well as secondary targets of the drug is unknown. The other actions and consequences of the drug may be beneficial or harmful. The identification of the full biological pathway of action of drugs or drug candidates is therefore a problem of commercial and human importance. Global gene expression profiling would provide a fast and inexpensive approach to characterizing drug activities and cellular pathways affected by drugs.

One way of achieving this is to measure expression levels of many genes expressed in particular tissues or cells at a particular time on a large or small scale. The use of DNA microarrays and other technological advances make such analyses available.

DNA microarrays are based on nucleic acids attached to a solid support. Nucleic acid sequences (cDNA or synthetic oligonucleotides for example) are attached to the solid support in grids and a pool of labeled RNA or cDNA from cell(s) or tissue(s) are hybridized. The intensity of the hybridization signal at each grid is measured and provide an estimate of the level expression of the genes. Nucleic acid microarrays based on oligonucleotides attached to a glass surface covering around 30,000 unique gene sequences ordered in high density on small slides (i.e. approximately one third to one fourth of all genes) are now available from commercial sources, such as Affymetrix. Thus, some microarrays are based on a high capacity system to monitor the expression of many genes in parallel with high sensitivity.

A number of alternative methods for detecting and quantification of gene expression are available. These include for instance Northern blot analysis, S1 nuclease protection assay, serial analysis of gene expression (SAGE) and sequencing of cDNA libraries. However, these are lower-throughput approaches.

The Celera GeneTag technology quantitatively measures the expression levels of virtually all RNA transcripts in a cell or tissue, whether previously known or uncharacterized. This allows simultaneous monitoring of known genes, uncharacterized genes and discovery of novel genes, saving significant time and costs relative to sequencing or other chip-based strategies. GeneTag technology provides this information within a biological context specific to the biological pathway, disease model, or drug response being investigated.

The GeneTag process is based on the principle that unique PCR fragments are generated for each cDNA. The fragments are separated by fluorescent capillary electrophoresis, then size-called and quantitated using Celera's proprietary algorithms. The amount of a specific mRNA is then determined by the fluorescent intensity of its cognate PCR fragment. Using Celera's proprietary GeneTag database, the cDNA fragment peaks are matched with their corresponding gene names. In this methodology, total RNA is isolated from the cell line(s) or tissues of interest. The GeneTag process requires at least 200 μg of total RNA.

Complementary DNA is prepared from the total RNA samples then restricted twice in a stepwise fashion. 3′-end capture is used after each digest to isolate the fragment of interest. Using this method, adapters are ligated to both ends of the fragment to serve as PCR primer sites. Thus, multiple fragments are potentially prepared for each gene. The adapter-ligated cDNA samples are amplified using a set of primers, which have two selective bases on each end. Combinations of these four bases yield a total of 128 unique PCR primer pairs. The 128 PCR reactions from each sample are analyzed individually by capillary electrophoresis, one reaction per capillary plus an internal-lane standard. Each gene presents one unique fragment that can be “binned” based on its size (bp) and the specific primer pair used to generate it. This binning process enables rapid data analysis and gene identification.

Celera's proprietary software assigns sizing and quantitation measures to each peak in the electropherogram. Internal size standards allow direct comparison of electropherograms from treated samples and controls.

All 128 electropherograms from both the treated samples and the control samples are analyzed and compared automatically. Peaks (cDNA fragments) exhibiting a statistically significant difference between sample and control are flagged and quantitated.

Another method described in U.S. Pat. Nos. 6,010,850 and 5,712,126 uses a Y-shaped adaptor to suppress non-3′fragments in the PCR. cDNA is digested with a restriction enzyme and ligated to a Y-shaped adapter. The Y-shaped adapter enables selective amplification of 3′-fragments. However, since the entire pool of cDNA is present, there are numerous opportunities for primers to hybridize non-specifically.

Digital Gene Technologies (La Jolla, Calif.) provides display of unique 3′-fragments. The method (U.S. Pat. No. 5,459,037) involves isolating and subcloning 3′-fragments, growing the subcloned fragments as a library in E. coli, extracting the plasmids, converting the inserts to cRNA and then back to DNA and then PCR amplifying. Both the above and this method is based on the use of a multiplex PCR (i.e. specific primers each protruding a few bases into unknown sequence; those bases varied across multiple reactions; each such reaction analyzed separately on a gel or capillary) to split the reaction in enough parts to be able to separate most bands from each other. This protocol achieves the objective of requiring relatively small amount of starting material while still purifying 3′ fragments, allowing a more stringent PCR.

A further method (WO 97/29211) describes profiling complementary DNA prepared from the total RNA sample, by digesting with a single restriction enzyme. Adaptors are hybridized to both ends of the fragments, after which the fragments are amplified using primer DNA sequences having one, two or three nucleotides hybridizing specifically to a subset of the complementary DNA molecules. Increasing the number of specific nucleotides increases the number of subdivisions. However, mismatching of primers can occur, decreasing the accuracy of fragment identification. WO 97/29211 describes a specific process which can be used to reduce mismatching. In the early stages of amplification a primer is used which comprises a single specific base; subsequently, in later cycles, primers with two specific bases are introduced, so as to progressively increase selectivity.

WO 99/42610 discloses an approach in which some degree of subdivision is achieved by the adaptors themselves. The initial restriction digestion is carried out with an enzyme which cuts at a site distinct from its recognition site (a Type IIS enzyme), and which thus leaves variable a overhang depending on the sequence of the target cDNA. Adaptors with variable sequences can then be ligated to these overhangs, thus subdividing the reaction.

The process of understanding medical events and interventions and developing therapeutics is known to be costly and time consuming. Development and administration of a drug that is ineffective results in wasted cost and time during which patients' conditions may significantly worsen. Also, administration of a drug to individuals in whom the drug would not be tolerated could result in a direct worsening of a patient's condition and could even result in a patient's death.

The time and expense of understanding medical events and interventions and developing therapeutics can be shortened by considering RNA expression, the levels of genomic RNA circulating in the blood stream, as indicative or predictive of future physiological conditions. For example, rising levels of RNA expressions associated with diabetes can be measured periodically in a grossly overweight individual to determine if that individual is more or less likely to later develop the disease. This analysis can be used to suggest earlier medical intervention or to help prevent unnecessary intervention.

Time-series quantitative gene expression analysis can be done without consideration to DNA sequence variation in the individual, and does not concern methods for identifying and exploiting gene sequence variances that account for interpatient variation in drug response, particularly interpatient variation attributable to pharmacokinetic factors and interpatient variation in drug tolerability or toxicity.

Adverse drug reactions are a principal cause of the low success rate of drug development programs (less than one in four compounds that enters human clinical testing is ultimately approved for use by the U.S. Food and Drug Administration (FDA)). Drug-induced disease or toxicity presents a unique series of challenges to drug developers, as these reactions are often not predictable from preclinical studies and may not be detected in early clinical trials involving small numbers of subjects. When such effects are detected in later stages of clinical development they often result in termination of a drug development program. When a drug is approved despite some toxicity, its clinical use is frequently severely constrained by the possible occurrence of adverse reactions in even a small group of patients. The likelihood of such a compound becoming first line therapy is small (unless there are no competing products). Clinical trials that use this invention may allow for improved predictions of possible toxic reactions in studies involving a small number of subjects. The methods of this invention offer a quickly derived prediction of likely future toxic effects of an intervention.

Absorption is the first pharmacokinetic parameter to consider when determining variation in drug response. The actual effects of absorption on an individual or group of individuals may be quickly determined using this invention.

Once a drug or candidate therapeutic intervention is absorbed, injected or otherwise enters the bloodstream it is distributed to various biological compartments via the blood. The drug may exist free in the blood, or, more commonly, may be bound with varying degrees of affinity to plasma proteins. One classic source of variation in drug response is attributable to amino acid polymorphisms in serum albumin, which affect the binding affinity of drugs such as warfarin. Consequent variation in levels of free warfarin has a significant effect on the degree of anticoagulation. From the blood a compound diffuses into and is retained in interstitial and cellular fluids of different organs to different degrees. The invention allows for use of genetic expressions to be used instead of measurements of the proteins reducing the time and complexity of measurements.

Once absorbed by the gastrointestinal tract, compounds encounter detoxifying and metabolizing enzymes in the tissues of the gastrointestinal system. Many of these enzymes are known to be polymorphic in man and account for well studied variation in pharmacokinetic parameters of many drugs. Subsequently compounds enter the hepatic portal circulation in a process commonly known as first pass. The compounds then encounter a vast array of xenobiotic detoxifying mechanisms in the liver, including enzymes that are expressed solely or at high levels only in liver. These enzymes include the cytochrome P450s, glucuronlytransferases, sulfotransferases, acetyltransferases, methyltransferases, the glutathione conjugating system, flavine monooxygenases, and other enzymes known in the art. The invention allows for quick measurement of metabolic effects.

Biotransformation reactions in the liver often have the effect of converting lipophilic compounds into hydrophilic molecules that are then more readily excreted. Variation in these conjugation reactions may affect half-life and other pharmacokinetic parameters. It is important to note that metabolic transformation of a compound not infrequently gives rise to a second or additional compounds that have biological activity greater than, less than, or different from that of the parent compound. Metabolic transformation may also be responsible for producing toxic metabolites. The invention allows for quick identification of biotransformation reactions.

Genomic expressions can be a precursor to medical events such as clinical responses. The method of the present invention allows for a prediction of clinical responses on an individual or generally across a population due to an event or intervention. A “Medical Event” is any occurrence that may result in death, may be life-threatening, may require hospitalization, or prolongation of existing hospitalization, may result in persistent or significant disability/incapacity, may be a congenital anomaly/birth defect, may require surgical or non-surgical intervention to prevent one or more of the outcomes listed in this definition, may result in a change in clinical symptoms, or otherwise may result in change in the health of an individual or group of individuals whether naturally or as a result of human intervention.

Different events or interventions may present different responses in gene expression within a subject or between subjects. The invention allows the gene expression responses from differing interventions to be compared to help determine relative effectiveness and toxicity among different interventions and medical events and interventions, including those described in Behrman: Nelson Textbook of Pediatrics, Braunwald: Heart Disease: A Textbook of Cardiovascular Medicine, Brenner: Brenner & Rector's The Kidney, Canale: Campbell's Operative Orthopaedics, Cotran: Robbins Pathologic Basis of Disease, Cummings et al: Otolaryngology—Head and Neck Surgery, DeLee: DeLee and Drez's Orthopaedic Sports Medicine, Duthie: Practice of Geriatric, Feldman: Sleisenger & Fordtran's Gastrointestinal and Liver Disease, Ferri: Ferri's Clinical Advisor, Ferri: Practical Guide to the Care of the Medical Patient, Ford: Clinical Toxicology, Gabbe: Obstetrics: Normal and Problem Pregnancies, Goetz: Textbook of Clinical Neurology, Goldberger: Clinical Electrocardiography, Goldman: Cecil Textbook of Medicine, Grainger: Grainger & Allison's Diagnostic Radiology, Habif: Clinical Dermatology: Color Guide to Diagnosis and Therapy, Hoffman: Hematology: Basic Principles and Practice, Jacobson: Psychiatric Secrets, Johns Hopkins: The Harriet Lane Handbook, Larsen: Williams Textbook of Endocrinology, Long: Principles and Practices of Pediatric Infectious Disease, Mandell: Principles and Practice of Infectious Diseases, Marx: Rosen's Emergency Medicine: Concepts and Clinical Practice, Middleton: Allergy: Principles and Practice, Miller: Anesthesia, Murray & Nadel: Textbook of Respiratory Medicine, Noble: Textbook of Primary Care Medicine, Park: Pediatric Cardiology for Practitioners, Pizzomo: Textbook of Natural Medicine, Rakel: Conn's Current Therapy, Rakel: Textbook of Family Medicine, Ravel: Clinical Laboratory Medicine, Roberts: Clinical Procedures in Emergency Medicine, Ruddy: Kelley's Textbook of Rheumatology, Ryan: Kistner's Gynecology and Women's Health Townsend: Sabiston Textbook of Surgery, Yanoff: Opthalmology, and Walsh: Campbell's Urology.

One of skill in the art will appreciate that in order to measure the transcription level (and thereby the expression level) of a gene or genes, it is desirable to provide a nucleic acid sample comprising mRNA transcript(s) of the gene or genes, or nucleic acids derived from the mRNA transcript(s). As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, suitable samples include, but are not limited to, mRNA transcripts of the gene or genes, cDNA reverse transcribed from the mRNA, cRNA transcribed from the cDNA, DNA amplified from the genes, RNA transcribed from amplified DNA, and the like. Genes are selected for monitoring either by statistical analysis of data provided by microarray or other quantitative gene techniques. Genes may also be selected for monitoring based on licensed or publicly available information.

Typically the genes are amplified by methods of primer directed amplification such as polymerase chain reaction (PCR) (U.S. Pat. No. 4,683,202 (1987, Mullis, et al.) and U.S. Pat. No. 4,683,195 (1986, Mullis, et al.), ligase chain reaction (LCR) (Tabor et al., 82 PROC. ACAD. SCI. U.S.A., 1074-1078 (1985)) or strand displacement amplification (Walker et al., 89 PROC. NATL. ACAD. SCI. U.S.A., 392, (1992) for example.

Probes bearing a signal generating label are synthesized. Probes may be randomly generated or may be synthesized based on the sequence of specific open reading frames. Probes useful in the present invention include, but are not limited to, single stranded nucleic acid sequences which are complementary to the nucleic acid sequences to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Signal generating labels that may be incorporated into the probes are well known in the art. For example labels may include but are not limited to fluorescent moieties, chemiluminescent moieties, particles, enzymes, radioactive tags, or light emitting moieties or molecules, where fluorescent moieties are preferred. Most preferred are fluorescent dyes capable of attaching to nucleic acids and emitting a fluorescent signal. A variety of dyes are known in the art such as fluorescein, Texas red, and rhodamine. Preferred in the present invention are the mono reactive dyes cy3 (146368-16-3) and cy5 (146368-14-1) both available commercially (i.e. Amersham Pharmacia Biotech, Arlington Heights, Ill.). Suitable dyes are discussed in U.S. Pat. No. 5,814,454 hereby incorporated by reference.

Labels may be incorporated by any of a number of means well known to those of skill in the art. However, in one embodiment, the label is simultaneously incorporated during the amplification step in the preparation of the probe nucleic acids. Thus, for example, polymerase chain reaction (PCR) with labeled primers or labeled nucleotides will provide a labeled amplification product. In another preferred embodiment, reverse transcription or replication, using a labeled nucleotide (e.g. dye-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the synthesis is completed. Means of attaching labels to nucleic acids are well known to those of skill in the art and include, for example nick translation or end-labeling (e.g. with a labeled RNA) by kinase-reacting the nucleic acid and subsequent attachment (ligation) of a nucleic acid linker joining the sample nucleic acid to a label (e.g., a fluorophore).

Following incorporation of the label into the probe the probes are then hybridized to the micro-array using standard conditions where hybridization results in a double stranded nucleic acid, generating a detectable signal from the label at the site of capture reagent attachment to the surface. Typically the probe and array must be mixed with each other under conditions which will permit nucleic acid hybridization. This involves contacting the probe and array in the presence of an inorganic or organic salt under the proper concentration and temperature conditions. The probe and array nucleic acids must be in contact for a long enough time that any possible hybridization between the probe and sample nucleic acid may occur. The concentration of probe or array in the mixture will determine the time necessary for hybridization to occur. The higher the probe or array concentration the shorter, the hybridization incubation time needed. Optionally a chaotropic agent may be added. The chaotropic agent stabilizes nucleic acids by inhibiting nuclease activity. Furthermore, the chaotropic agent allows sensitive and stringent hybridization of short oligonucleotide probes at room temperature [Van Ness and Chen, 19 NUCL. ACIDS RES. 5143-5151 (1991)]. Suitable chaotropic agents include guanidinium chloride, guanidinium thiocyanate, sodium thiocyanate, lithium tetrachloroacetate, sodium perchlorate, rubidium tetrachloroacetate, potassium iodide, and cesium trifluoroacetate, among others. Typically, the chaotropic agent will be present at a final concentration of about 3 M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers, such as sodium citrate, Tris-HCl, PIPES or HEPES (pH range about 6-9), about 0.05 to 0.2% detergent, such as sodium dodecylsulfate, or between 0.5-20 mM EDTA, FICOLL (Pharmacia, Inc.) (about 300-500 kilodaltons), polyvinylpyrrolidone (about 250-500 kdal), and serum albumin. Also included in the typical hybridization solution will be unlabeled carrier nucleic acids from about 0.1 to 5 mg/mL, fragmented nucleic DNA, e.g., calf thymus or salmon sperm DNA, or yeast RNA, and optionally from about 0.5 to 2% wt./vol. glycine. Other additives may also be included, such as volume exclusion agents which include a variety of polar water-soluble or swellable agents, such as polyethylene glycol, anionic polymers such as polyacrylate or polymethylacrylate, and anionic saccharidic polymers, such as dextran sulfate. Methods of optimizing hybridization conditions are well known to those of skill in the art (see, e.g., Laboratory Techniques in Biochemistry and Molecular Biology, Volume 24: Hybridization with Nucleic Acid Probes, (P. Tijssen, Ed. Elsevier, N.Y., (1993)) and Maniatis, supra.

The basis of gene expression profiling via micro-array technology relies on comparing nucleic acid molecules from a subject under a variety of conditions that include pre-treatment or baseline conditions, or events that result in alteration of the genes expressed. Within the context of the present invention a subject may be exposed to a variety of medical events or interventions, including treatments, conditions or stresses that resulted in the alteration of gene expression. Typical stresses that result in an alteration in gene expression profile include, but are not limited to, conditions altering the growth of a cell, exposure to mutagens, drugs, chemicals, antibiotics, UV light, gamma-rays, x-rays, phage, macrophages, organic chemicals, inorganic chemicals, environmental pollutants, heavy metals, changes in temperature, changes in pH, conditions producing oxidative damage, DNA damage, anaerobiosis, depletion or addition of nutrients, and addition of a growth inhibitor. Untreated cells are used for generation of “control” or “baseline” arrays and treated or disease cells are used to generate an “experimental,” “stressed,” or “induced” arrays for comparison.

Definitions: Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a device” includes a combination of two or more such devices, reference to “a gene fusion construct” includes mixtures of constructs, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, currently preferred materials and methods are described herein.

In describing and claiming the present invention, the following terminology will be used in accordance with the definitions set out below.

The term “absolute abundance” or “absolute gene expression levels” refers to the amount of a particular species (e.g., gene expression product) present in a sample.

The term “amplified product” refers to a nucleic acid generated by any method of nucleic acid amplification.

The terms “array”, “polynucleotide array”, “microarray”, and “probe array” all refer to a surface on which is attached or deposited a molecule capable of specifically binding to a polynucleotide of a given sequence. Typically the molecule will be a polynucleotide having a sequence complimentarity to the polynucleotide to be detected, and capable of hybridizing to it.

The term “attenuation” refers to a method of reducing the signal intensities of extremely abundant reaction products in a multiplex, such that the signals from all products of a multiplex set of products fall within the dynamic range of the detection platform used for the assay.

The term “cDNA” refers to complementary or “copy” DNA. Generally cDNA is synthesized by a DNA polymerase using any type of RNA molecule (e.g., typically mRNA) as a template. Alternatively, the cDNA can be obtained by directed chemical syntheses.

The term “chemical treatment” refers to the process of exposing a cell, cell line, tissue, subject or organism to a chemical or biochemical compound (or library of compounds) that has/have the potential to alter its gene expression profile.

The term “complementary” refers to nucleic acid sequences capable of base-pairing according to the standard Watson-Crick complementary rules, or being capable of hybridizing to a particular nucleic acid segment under relatively stringent conditions. Nucleic acid polymers are optionally complementary across only portions of their entire sequences.

The term “environmental stress” refers to an externally applied factor or condition that may cause an alteration in the gene expression profile of a cell.

The term “gene” refers to a nucleic acid sequence encoding a gene product. The gene optionally comprises sequence information required for expression of the gene (e.g., promoters, enhancers, etc.). The term “genomic” relates to the genome of an organism.

The term “gene expression” refers to transcription of a gene into an RNA product, and optionally to translation into one or more polypeptide sequences.

The term “gene expression data” refers to one or more sets of data that contain information regarding different aspects of gene expression. The data set optionally includes information regarding: the presence of target-transcripts in cell or cell-derived samples; the relative and absolute abundance levels of target transcripts; the ability of various treatments to induce expression of specific genes; and the ability of various treatments to change expression of specific genes to different levels.

The term “gene expression profile” refers to a representation of the expression level of a plurality of genes without (i.e. baseline or control), or in response to, a selected expression condition (for example, incubation of the presence of a standard compound or test compound at one or several timepoints). Gene expression can be expressed in terms of an absolute quantity of mRNA transcribed for each gene, as a ratio of mRNA transcribed in a test cell as compared with a control cell, and the like. It also refers to the expression of an individual gene and of suites of individual genes in a subject.

The term “growth-altering environment” refers to energy, chemicals, or living things that have the capacity to modulate cell growth or function. Inhibitory agents may include but are not limited to mutagens, drugs, antibiotics, UV light, gamma-rays, x-rays, temperature, virus, T-cells, macrophages, organic chemicals and inorganic chemicals.

The term “high throughput format” refers to analyzing more than about 10 samples per hour, about 50 or more samples per hour, about 100 or more samples per hour, or about 250, about 500, about 1000 or more samples per hour.

The term “hybridization” refers to duplex formation between two or more polynucleotides, e.g., to form a double-stranded nucleic acid. The ability of two regions of complementarity to hybridize and remain together depends of the length and continuity of the complementary regions, and the stringency of hybridization conditions.

The term “insult” or “environmental insult” refers to any substance or environmental change that results in an alteration of normal cellular metabolism in a cell, organism, subject or population of cells. Environmental insults may include, but are not limited to, chemicals, environmental pollutants, heavy metals, changes in temperature, changes in pH, as well as agents producing oxidative damage, DNA damage, anaerobiosis, and changes in nutrient availability or pathogenesis.

The term “label” refers to any detectable moiety. A label may be used to distinguish a particular nucleic acid from others that are unlabeled, or labeled differently, or the label may be used to enhance detection.

The term “medical event” refers to any occurrence that may result in death, may be life-threatening, may require hospitalization, or prolongation of existing hospitalization, may result in persistent or significant disability/incapacity, may be a congenital anomaly/birth defect, may require surgical or non-surgical intervention to prevent one or more of the outcomes listed in this definition, may result in a change in clinical symptoms, or otherwise may result in change in the health of an individual or group of individuals whether naturally or as a result of human intervention.

The terms “microplate,” “culture plate,” and “multiwell plate” interchangeably refer to a surface having multiple chambers, receptacles or containers and generally used to perform a large number of discreet reactions simultaneously.

The term “miniaturized format” refers to procedures or methods conducted at submicroliter volumes, including on both microfluidic and nanofluidic platforms.

The term “multiplex reaction” refers to a plurality of reactions conducted simultaneously in a single reaction mixture.

The term “multiplex amplification” refers to a plurality of amplification reactions conducted simultaneously in a single reaction mixture.

The term “nucleic acid” refers to a polymer of ribonucleic acids or deoxyribonucleic acids, including RNA, mRNA, rRNA, tRNA, small nuclear RNAs, cDNA, DNA, PNA, or RNA/DNA copolymers. Nucleic acid may be obtained from a cellular extract, genomic or extragenomic DNA, viral RNA or DNA, or artificially/chemically synthesized molecules.

The term “platform” refers to the instrumentation method used for sample preparation, amplification, product separation, product detection, or analysis of data obtained from samples.

The term “primer” refers to any nucleic acid that is capable of hybridizing to a complementary nucleic acid molecule, and that optionally provides a free 3′ hydroxyl terminus which can be extended by a nucleic acid polymerase.

The term “reference sequence” refers to a nucleic acid sequence serving as a target of amplification in a sample that provides a control for the assay. The reference may be internal (or endogenous) to the sample source, or it may be an externally added (or exogenous) to the sample. An external reference may be, for example, either RNA, added to the sample prior to reverse transcription, or DNA (e.g., cDNA), added prior to PCR amplification.

The term “relative abundance” or “relative gene expression levels” refers to the abundance of a given species relative to that of a second species. Optionally, the second species is a reference sequence.

The term “RNA” refers to a polymer of ribonucleic acids, including RNA, mRNA, rRNA, tRNA, and small nuclear RNAs, as well as to RNAs that comprise ribonucleotide analogues to natural ribonucleic acid residues, such as 2-O-methylated residues.

The term “separation system” refers to any of a set of methodologies that can be employed to effect a size separation of the products of a reaction.

The term “size separation” refers to physical separation of a complex mixture of species into individual components according to the size of each species.

The term “stress” or “environmental stress” refers to the condition produced in a cell as the result of exposure to an environmental insult.

The term “stress gene” refers to any gene whose transcription is increased or decreased as a result of environmental stress or by the presence of an environmental insult.

The term “stress response” refers to the cellular response to an environmental insult.

The term “target,” “target sequence,” or “target gene sequence” refers to a specific nucleic acid sequence, the presence, absence or abundance of which is to be determined.

In a preferred embodiment of the invention, it is a unique sequence within the mRNA of an expressed gene.

The term “target-specific primer” refers to a primer capable of hybridizing with its corresponding target sequence. Under appropriate conditions, the hybridized primer can prime the replication of the target sequence.

The term “template” refers to any nucleic acid polymer that can serve as a sequence that can be copied into a complementary sequence by the action of, for example, a polymerase enzyme.

The term “transcription” refers to the process of copying a DNA sequence of a gene into an RNA product, generally conducted by a DNA-directed RNA polymerase using the DNA as a template.

The term “treatment” refers to the process of subjecting one or more cells, cell lines, tissues, or organisms to a condition, substance, or agent (or combinations thereof) that may cause the cell, cell line, tissue or organism to alter its gene expression profile. A treatment may include a range of chemical concentrations and exposure times, and replicate samples may be generated.

The term “universal primer” refers to a replication primer comprising a universal sequence.

The term “universal sequence” refers to a sequence contained in a plurality of primers, but preferably not in a complement to the original template nucleic acid (e.g., the target sequence), such that a primer composed entirely of universal sequence is not capable of hybridizing with the template.

Gene Expression as a Marker of the Biological State of a Subject Transcription of genes into RNA is a critical early step in gene expression. Consequently, the coordinated activation or suppression of transcription of particular genes is an important component of the overall regulation of expression. A variety of well-developed techniques have been established that provide ways to analyze and quantitate gene transcription.

Some of the earliest and well known methods are based on detection of a label in RNA hybrids or protection of RNA from enzymatic degradation (see, for example, Current Protocols in Molecular Biology (F. M. Ausubel et al., Eds.), Current Protocols, a joint venture between Greene Publishing Associates, Inc. and John Wiley & Sons, Inc., supplemented through 1999). Methods based on detecting hybrids include northern blots and slot/dot blots. These two techniques differ in that the components of the sample being analyzed are resolved by size in a northern blot prior to detection, which enables identification of more than one species simultaneously. Slot blots are generally carried out using unresolved mixtures or sequences, but can be easily performed in serial dilution, enabling a more quantitative analysis.

In situ hybridization is a technique that monitors transcription by directly visualizing RNA hybrids in the context of a whole cell. This method provides information regarding subcellular localization of transcripts, and can be quantitative as well.

Techniques to monitor RNA that make use of protection from enzymatic degradation include S1 analysis and RNAse protection assays (RPAs). Both of these assays employ a labeled nucleic acid probe, which is hybridized to the RNA species being analyzed, followed by enzymatic degradation of single-stranded regions of the probe. Analysis of the amount and length of probe protected from degradation is used to determine the quantity and endpoints of the transcripts being studied. Although both methods can yield quantitative results, they are time-consuming and cumbersome, making them poor candidates for a high-throughput, low cost general assay for gene expression.

Other assays developed for monitoring transcription make use of cDNA derived from mRNA. Because the material analyzed is DNA, these assays are less sensitive to degradation, and also provide partial and/or full clones with which to localize and clone genes or coding sequences of interest. Methods include sequencing cDNA inserts of an expressed sequence tag (EST) clone library (Adams et al., 252 SCIENCE 1651-1656 (1991)), which may be coupled with subtractive hybridization to improve sensitivity (Sagerstrom et al., 66 ANNUL REV. BIOCHEM. 751-783 (1997)), and serial analysis of gene expression (“SAGE”, described in U.S. Pat. No. 5,866,330 to Kinzler et al.; Velculescu et al., 270 SCIENCE 484-487 (1995)); and Zhang et al., 276 SCIENCE 1268-1272 (1997)). Both of these methods have been useful for identification of novel, differentially expressed genes. These methodologies yield untargeted information, i.e., they survey the whole spectrum of mRNA in a sample rather than focusing on a predetermined set. This method may provide a subset of genes whose expression level increases or decreases in response to an event or as a result of having a certain genetic background.

Reverse transcriptase-mediated PCR (RT-PCR) gene expression assays are directed at specified target gene products, overcoming some of the shortcomings described above. These assays are derivatives of PCR in which amplification is preceded by reverse transcription of mRNA into cDNA. Because the mRNA is amplified, this type of assay can detect transcripts of very low abundance; however, the assay is not quantitative. Adaptations of this assay, called competitive RT-PCR (Becker-Andre and Hahlbrock, 17 NUCLEIC ACIDS RES. 9437-9446 (1989); Wang et al., 86 PROC. NATL. ACAD. SCI. USA 9717-9721 (1989); Gilliland et al., 87 PROC. NATL. ACAD. SCI. USA 2725-2729 (1990)) have been developed that are more quantitative. In these assays, a known amount of exogenous template is added to the reaction mixture, to compete with the target for amplification. The exogenous competitor is titrated against the target, allowing for quantitation of a specified cDNA in the sample by comparing the amplification of both templates within the same reaction mixture. Because titration is required to generate quantitative data, multiple reactions are required for each analysis. While this type of assay is very sensitive and quantitative, these assays require multiple steps in development, execution, and analysis, making them very time-consuming, cumbersome, and expensive.

In order to increase the throughput of the RT-PCR assay, Su et al. (22 BIOTECHNIQUES 1107-1113 (1997)) combined microplate-based RNA extraction with multiplexed RT-PCR. With this method, they demonstrated simultaneous analysis of three different target mRNAs amplified from samples prepared from a 96 well microplate. However, changes in gene expression were only presented qualitatively.

Other methods for targeted mRNA analysis include differential display reverse transcriptase PCR (DDRT-PCR) and RNA arbitrarily primed PCR (RAP-PCR) (see U.S. Pat. No. 5,599,672; Liang and Pardee, 257 SCIENCE 967-971 (1992); Welsh et al., 20 NUCLEIC ACIDS RES. 4965-4970 (1992)). Both methods use random priming to generate RT-PCR fingerprint profiles of transcripts in an unfractionated RNA preparation. The signal generated in these types of analyses is a pattern of bands separated on a sequencing gel. Differentially expressed genes appear as changes in the fingerprint profiles between two samples, which can be loaded in separate wells of the same gel. This type of readout allows identification of both up- and down-regulation of genes in the same reaction, appearing as either an increase or decrease in intensity of a band from one sample to another.

The TaqMan assay (Livak et al., 4 PCR METHODS APPL. 357-362 (1995)) is a quenched fluorescent dye system for quantitating targeted mRNA levels in a complex mixture. The assay has good sensitivity and dynamic range, and yields quantitative results.

Nucleic acid microarrays have been developed recently, which have the benefit of assaying for sample hybridization to a large number of probes in a highly parallel fashion. They can be used for quantitation of mRNA expression levels, and dramatically surpass the above mentioned techniques in terms of multiplexing capability. These arrays comprise short DNA sequences, PCR products, or mRNA isolates fixed onto a solid surface, which can then be used in a hybridization reaction with a target sample, generally a whole cell extract (see, for example, U.S. Pat. Nos. 5,143,854 and 5,807,522; Fodor et al., 251 SCIENCE 767-773 (1991); and Schena et al., 270 SCIENCE 467-470 (1995)). Microarrays can be used to measure the expression levels of several thousands of genes simultaneously, generating a gene expression profile of the entire genome of relatively simple organisms. Each reaction, however, is performed with a single sample against a very large number of gene probes. As a consequence, microarray technology does not facilitate high throughput analysis of very large numbers of unique samples against an array of known probes.

The present invention addresses the need for obtaining gene expression detection and quantitation in an individual or group of individuals by providing novel methods for analyzing gene expression, systems for implementing these techniques, compositions for preparing a plurality of amplification products from a plurality of mRNA target sequences, and related pools of amplification products. The methods of the present invention include the steps of (a) obtaining a plurality of target RNA or cDNA sequences; (b) amplifying the target sequences using a plurality of target-specific primers and one or more universal primers; (c) detecting the one or more members of the plurality of amplification products, thereby generating a set of gene expression data; (d) storing the data in a database; and (e) performing a comparative analysis on the set of gene expression data, thereby analyzing the gene expression. The methods of the invention are highly sensitive; have a wide dynamic range; are rapid and inexpensive; have a high throughput; and allow the simultaneous differential analysis of a defined set of genes or of the entire genome of a subject. The methods, compositions and kits of the invention also provide tools for gene expression data collection and relational data analysis.

Methods for Quantitating Gene Expression Levels The controlled expression of particular genes or groups of genes in a cell is the molecular basis for regulation of biological processes and, ultimately, for the physiological or pathological state of the cell. Knowledge of the “expression profile” of a cell is of key importance for answering many biological questions, including the nature and mechanism of cellular changes, or the degree of differentiation of a cell, organ, or organism. Furthermore, the factors involved in determining the expression profile may lead to the discovery of cures that could reverse an adverse pathological or physiological condition. A defined set of genes can be demonstrated to serve as indicators of a particular state of a cell, and can therefore serve as a model for monitoring the cellular profile of gene expression in that state.

The pharmaceutical drug discovery process has traditionally been dominated by biochemical and enzymatic studies of a designated pathway. Although this approach has been productive, it is very laborious and time-consuming, and is generally targeted to a single gene or defined pathway. Molecular biology and the development of gene cloning have dramatically expanded the number of genes that are potential drug targets, and this process is accelerating rapidly as a result of the progress made in sequencing the human genome. In addition to the growing set of available genes, techniques such as the synthesis of combinatorial chemical libraries have created daunting numbers of candidate drugs for screening. In order to capitalize on these available materials, methods are needed that are capable of extremely fast and inexpensive analysis of gene expression levels.

The present invention provides novel methods for the analysis of changes in expression levels of a set of genes. These methods include providing a plurality of target sequences, which are then analyzed simultaneously in a multiplexed reaction. Multiplexing the analysis improves the accuracy of quantitation; for example, signals from one or more target genes can be compared to an internal control. Multiplexing also reduces the time and cost required for analysis. Thus, the methods of the present invention provide for rapid generation of a differential expression profile of a defined set of genes, through the comparison of data from multiple reactions.

The methods of the present invention include the steps of (a) obtaining a plurality of target nucleic acid sequences, generally cDNA sequences; (b) multiplex amplifying the target sequences using a plurality of target-specific primers and one or more universal primers; (c) separating one or more members of the resulting plurality of amplification products; (d) detecting the one or more members of the plurality of amplification products, thereby generating a set of gene expression data; (e) storing the data in a database; and (f) performing a comparative analysis on one or more components of the set of gene expression data, thereby analyzing the gene expression. In an alternative embodiment, the methods of the present invention include the steps of obtaining cDNA from a plurality of samples for a plurality of target sequences; performing a plurality of multiplexed amplifications of the target sequences, thereby producing a plurality of multiplexed amplification products; pooling the plurality of multiplexed amplification products; separating the plurality of multiplexed amplification products; detecting the plurality of multiplexed amplification products, thereby generating a set of gene expression data; storing the set of gene expression data in a database; and performing a comparative analysis of the set of gene expression data. In yet another embodiment, the methods of the present invention include the steps of (a) obtaining cDNA from multiple samples; (b) amplifying a plurality of target sequences from the cDNA, thereby producing a multiplex of amplification products; (c) separating and detecting the amplification products using a high throughput platform, wherein detecting generates a set of gene expression data; (d) storing the set of gene expression data in a database; and (e) performing a comparative analysis of the set of gene expression data. Each aspect of these methods of the present invention is addressed in greater detail below.

Sources of Target Sequences Target sequences for use in the methods of the present invention are obtained from a number of sources. For example, the target sequences can be derived from subjects such as humans, animals, organisms, or from cultured cell lines. Cell types utilized in the present invention can be either prokaryotic or eukaryotic cell types and/or organisms, including, but not limited to, animal cells, plants, yeast, fungi, bacteria, viruses, and the like. Target sequences can also be obtained from other sources, for example, needle aspirants or tissue samples from an organism (including, but not limited to, mammals such as mice, rodents, guinea pigs, rabbits, dogs, cats, primates and humans; or non-mammalian animals such as nematodes, frogs, amphibians, various fishes such as the zebra fish, and other species of scientific interest), non-viable organic samples or their derivatives (such as a cell extract or a purified biological sample), or environmental sources, such as an air or water sample. Furthermore, target sequences can also be commercially or synthetically prepared, such as a chemical, phage, or plasmid library. DNA and/or RNA sequences are available from a number of commercial sources, including The Midland Certified Reagent Company (Midland, Tex.), The Great American Gene Company (Salt Lake City, Utah), ExpressGen Inc. (Chicago, Ill.), Operon Technologies Inc. (Alameda, Calif.) and many others.

Cell lines which can be used in the methods of the present invention include, but are not limited to, those available from cell repositories such as the American Type Culture Collection (Manassas, Va.), the World Data Center on Microorganisms (Mishima, Japan), European Collection of Animal Cell Culture (Salisbury, United Kingdom) and the Japanese Cancer Research Resources Bank (Osaka, Japan). These cell lines include, but are not limited to, the following cell lines: 293, 293Tet-Off, CHO-AA8 Tet-Off, MCF7, MCF7 Tet-Off, LNCap, T-5, BSC-1, BHK-21, Phinx-A, 3T3, HeLa, PC3, DU145, ZR 75-1, HS 578-T, DBT, Bos, CV1, L-2, RK13, HTTA, HepG2, BHK-Jurkat, Daudi, RAMOS, KG-1, K562, U937, HSB-2, HL-60, MDAHB231, C2C12, HTB-26, HTB-129, HPIC5, A-431, CRL-1573, 3T3L1, Cama-1, J774A.1, HeLa 229, PT-67, Cos7, OST7, HeLa-S, THP-1, and NXA. Additional cell lines for use in the methods and matrices of the present invention can be obtained, for example, from cell line providers such as Clonetics Corporation (Walkersville, Md.). Optionally, the plurality of target sequences are derived from cultured cells optimized for the analysis of a particular disease area of interest, e.g., cancer, inflammation, cardiovascular disease, diabetes, infectious diseases, proliferative diseases, an immune system disorder, or a central nervous system disorder.

A variety of cell culture media are described in The Handbook of Microbiological Media (Atlas and Parks Eds., CRC. Press, Boca Raton, Fla., 1993). References describing the techniques involved in bacterial and animal cell culture include Sambrook et al., Molecular Cloning—A Laboratory Manual, 2^{nd} Edition, Volumes 1-3 (Cold Spring Harbor Laboratory, Cold Spring Harbor, N.Y., 1989); Current Protocols in Molecular Biology (F. M. Ausubel et al., Eds., Current Protocols, (a joint venture between Greene Publishing Associates, Inc. and John Wiley & Sons, Inc., supplemented through 2000); Freshney, Culture of Animal Cells, a Manual of Basic Technique, Third Edition (Wiley-Liss, New York, 1994) and the references cited therein; Humason, Animal Tissue Techniques, Fourth Edition (W. H. Freeman and Company, New York, 1979); and Ricciardelli, et al., 25 IN VITRO CELL DEV. BIOL. 1016-1024 (1989). Information regarding plant cell culture can be found in Plant Cell and Tissue Culture in Liquid Systems (Payne et al., John Wiley & Sons, Inc. New York, N.Y., 1992); Plant Cell, Tissue and Organ Culture: Fundamental Methods (Gamborg and Phillips, Eds., Springer Lab Manual, Springer-Verlag, Berlin, 1995), and is also available in commercial literature such as the Life Science Research Cell Culture Catalogue (Sigma-Aldrich, Inc., St Louis, Mo., 1998) (Sigma-LSRCCC) and the Plant Culture Catalogue and supplement (Sigma-Aldrich, Inc., St Louis, Mo., 1997) (Sigma-PCCS).

In an exemplary embodiment of methods of the present invention, either primary or immortalized (or other) cell lines are grown in a master flask, then trypsinized (if they are adherent) and transferred to a 96-well plate, seeding each well at a density of 10⁴ to 10⁶ cells/well. If the gene expression profile in response to a chemical treatment is sought, the chemical agent of choice is prepared in a range of concentrations. After a time of recovery and growth as appropriate to the cell line, cells are exposed to the chemical for a period of time that will not adversely impact the viability of the cells. Preferably, assays include a range of chemical concentrations and exposure times, and would include replicate samples. After treatment, medium is removed and cells are immediately lysed.

In further embodiments of cell culture, formats other than a 96-well plate may be used. Other multiwell or microplate formats containing various numbers of wells, such as 6, 12, 48, 384, 1536 wells, or greater, are also contemplated. Culture formats that do not use conventional flasks, as well as microtiter formats, may also be used.

Treatment of Cells The cells lines or sources containing the target nucleic acid sequences, are optionally subjected to one or more specific treatments, or in the case of organisms, may already be in different pathological or physiological stages that induce changes in gene expression. For example, a cell or cell line can be treated with or exposed to one or more chemical or biochemical constituents, e.g., pharmaceuticals, pollutants, DNA damaging agents, oxidative stress-inducing agents, pH-altering agents, membrane-disrupting agents, metabolic blocking agent; a chemical inhibitors, cell surface receptor ligands, antibodies, transcription promoters/enhancers/inhibitors, translation promoters/enhancers/inhibitors, protein-stabilizing or destabilizing agents, various toxins, carcinogens or teratogens, characterized or uncharacterized chemical libraries, proteins, lipids, or nucleic acids. Optionally, the treatment comprises an environmental stress, such as a change in one or more environmental parameters including, but not limited to, temperature (e.g. heat shock or cold shock), humidity, oxygen concentration (e.g., hypoxia), radiation exposure, culture medium composition, or growth saturation. Alternatively, cultured cells may be exposed to other viable organisms, such as pathogens or other cells, to study changes in gene-expression that result from biological events, such as infections, or cell-cell interactions. Responses to these treatments may be followed temporally, and the treatment can be imposed for various times and at various concentrations. Target sequences can also be derived from cells or organisms exposed to multiple specific treatments as described above, either concurrently or in tandem (i.e., a cancerous tissue sample may be further exposed to a DNA damaging agent while grown in an altered medium composition).

RNA may be isolated from subjects prior to any treatment or appearance of symptoms in order to establish a baseline or control gene expression profile. This control or baseline is used to compare to a gene expression profile generated after a medical event or other occurrence that could result in a change in the gene expression profile. Occurrences that may result in a change in the gene expression profile include but are not limited to, treatment with a drug or drugs, a course of therapy, beginning or on-going dosing schedules, and amounts of a drug, trauma, progression of a pre-disease state, aging, drug withdrawal, weight loss, changes to circadian rhythm. Medical events and interventions include those described in Behrman: Nelson Textbook of Pediatrics, Braunwald: Heart Disease: A Textbook of Cardiovascular Medicine, Brenner: Brenner & Rector's The Kidney, Canale: Campbell's Operative Orthopaedics, Cotran: Robbins Pathologic Basis of Disease, Cummings et al: Otolaryngology—Head and Neck Surgery, DeLee: DeLee and Drez's Orthopaedic Sports Medicine, Duthie: Practice of Geriatric, Feldman: Sleisenger & Fordtran's Gastrointestinal and Liver Disease, Ferri: Ferri's Clinical Advisor, Ferri: Practical Guide to the Care of the Medical Patient, Ford: Clinical Toxicology, Gabbe: Obstetrics: Normal and Problem Pregnancies, Goetz: Textbook of Clinical Neurology, Goldberger: Clinical Electrocardiography, Goldman: Cecil Textbook of Medicine, Grainger: Grainger & Allison's Diagnostic Radiology, Habif: Clinical Dermatology: Color Guide to Diagnosis and Therapy, Hoffman: Hematology: Basic Principles and Practice, Jacobson: Psychiatric Secrets, Johns Hopkins: The Harriet Lane Handbook, Larsen: Williams Textbook of Endocrinology, Long: Principles and Practices of Pediatric Infectious Disease, Mandell: Principles and Practice of Infectious Diseases, Marx: Rosen's Emergency Medicine: Concepts and Clinical Practice, Middleton: Allergy: Principles and Practice, Miller: Anesthesia, Murray & Nadel: Textbook of Respiratory Medicine, Noble: Textbook of Primary Care Medicine, Park: Pediatric Cardiology for Practitioners, Pizzorno: Textbook of Natural Medicine, Rakel: Conn's Current Therapy, Rakel: Textbook of Family Medicine, Ravel: Clinical Laboratory Medicine, Roberts: Clinical Procedures in Emergency Medicine, Ruddy: Kelley's Textbook of Rheumatology, Ryan: Kistner's Gynecology and Women's Health Townsend: Sabiston Textbook of Surgery, Yanoff: Opthalmology, Walsh: Campbell's Urology.

RNA Isolation In some embodiments of the present invention, total RNA is isolated from samples for use as target sequences. Cellular samples are lysed once culture with or without the treatment is complete by, for example, removing growth medium and adding a guanidinium-based lysis buffer containing several components to stabilize the RNA. In some embodiments of the present invention, the lysis buffer also contains purified RNAs as controls to monitor recovery and stability of RNA from cell cultures. Examples of such purified RNA templates include the Kanamycin Positive Control RNA from Promega (Madison, Wis.), and 7.5 kb Poly(A)-Tailed RNA from Invitrogen (San Diego, Calif.). Lysates may be used immediately or stored frozen at, e.g., −80° C.

Optionally, total RNA is purified from cell lysates (or other types of samples) using silica-based isolation in an automation-compatible, 96-well format, such as the Rneasy® purification platform (Qiagen, Inc., Valencia, Calif.). Alternatively, RNA is isolated using solid-phase oligo-dT capture using oligo-dT bound to microbeads or cellulose columns. This method has the added advantage of isolating mRNA from genomic DNA and total RNA, and allowing transfer of the mRNA-capture medium directly into the reverse transcriptase reaction. Other RNA isolation methods are contemplated, such as extraction with silica-coated beads or guanidinium. Further methods for RNA isolation and preparation can be devised by one skilled in the art.

Alternatively, the methods of the present invention are performed using crude cell lysates, eliminating the need to isolate RNA. RNAse inhibitors are optionally added to the crude samples. When using crude cellular lysates, genomic DNA could contribute one or more copies of target sequence, depending on the sample. In situations in which the target sequence is derived from one or more highly expressed genes, the signal arising from genomic DNA may not be significant. But for genes expressed at very low levels, the background can be eliminated by treating the samples with DNAse, or by using primers that target splice junctions. For example, one of the two target-specific primers could be designed to span a splice junction, thus excluding DNA as a template. As another example, the two target-specific primers are designed to flank a splice junction, generating larger PCR products for DNA or unspliced mRNA templates as compared to processed mRNA templates. One skilled in the art could design a variety of specialized priming applications that would facilitate use of crude extracts as samples for the purposes of this invention.

Primer Design and Multiplex Strategies Multiplex amplification of the target sequence involves combining the plurality of target sequences with a plurality of target-specific primers and one or more universal primers, to produce a plurality of amplification products. A multiplex set of target sequences optionally comprises between about two targets and about 100 targets. In one embodiment of the present invention, the multiplex reaction includes at least 5 target sequences, but preferably at least ten targets or at least fifteen targets. Multiplexes of much larger numbers (e.g., about 20, about 50, about 75 and greater) are also contemplated.

In one embodiment of the methods of the present invention, at least one of the amplification targets in the multiplex set is a transcript that is endogenous to the sample and has been independently shown to exhibit a fairly constant expression level (for example, a “housekeeping” gene). The signal from this endogenous reference sequence provides a control for converting signals of other gene targets into relative expression levels. Optionally, a plurality of control mRNA targets/reference sequences that have relatively constant expression levels may be included in the multiplexed amplification to serve as controls for each other. Alternatively, a defined quantity of an exogenous purified RNA species is added to the multiplex reaction or to the cells, for example, with the lysis reagents. Almost any purified, intact RNA species can be used, e.g. the Kanamycin Positive Control RNA or the 7.5 kb Poly(A)-Tailed RNA mentioned previously. This exogenously-added amplification target provides a way to monitor the recovery and stability of RNA from cell cultures. It can also serve as an exogenous reference signal for converting the signals obtained from the sample mRNAs into relative expression levels. In still another embodiment, a defined quantity of a purified DNA species is added to the PCR to provide an exogenous reference target for converting the signals obtained from sample mRNA targets into relative expression levels.

In one embodiment of the present invention, once the targets that comprise a multiplex set are determined, primer pairs complementary to each target sequence are designed, including both target-specific and universal primers. This can be accomplished using any of several software products that design primer sequences, such as OLIGO (Molecular Biology Insights, Inc., CO), Gene Runner (Hastings Software Inc., New York), or Primer3 (The Whitehead Institute, Massachusetts).

Oligonucleotide primers are typically prepared by the phosphoramidite approach. In this automated, solid-phase procedure, each nucleotide is individually added to the 5′-end of the growing oligonucleotide chain, which is in turn attached at the 3′-end to a solid support. The added nucleotides are in the form of trivalent 3′-phosphoramidites that are protected from polymerization by a dimethoxytrityl (“DMT”) group at the 5′-position. After base induced phosphoramidite coupling, mild oxidation to give a pentavalent phosphotriester intermediate and DMT removal provides a new site for oligonucleotide elongation. These syntheses may be performed on, for example, a Perkin Elmer/Applied Biosystems Division DNA synthesizer. The oligonucleotide primers are then cleaved off the solid support, and the phosphodiester and exocyclic amino groups are deprotected with ammonium hydroxide.

Nucleic Acid Hybridization The length of complementary sequence between each primer and its binding partner (i.e. the target sequence or the universal sequence) should be sufficient to allow hybridization of the primer only to its target within a complex sample at the annealing temperature used for the PCR. A complementary sequence of, for example, about 15, 16, 17, 18, 19, 20, 21, 22, 23, 24, or 25 or more nucleotides is preferred for both the target-specific and universal regions of the primers. A particularly preferred length of each complementary region is about 20 bases, which will promote formation of stable and specific hybrids between the primer and target.

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Laboratory Techniques in Biochemistry and Molecular Biology—Hybridization with Nucleic Acid Probes, Part I, Chapter 2, “Overview of Principles of Hybridization and the Strategy of Nucleic Acid Probe Assays”, (Tijssen, Elsevier, New York, 1993), as well as in Ausubel, supra. (Hames and Higgins 1) Gene Probes 1, (Hames and Higgins, IRL Press at Oxford University Press, Oxford, England, 1995) and (Hames and Higgins 2) Gene Probes 2 (Hames and Higgins, IRL Press at Oxford University Press, Oxford, England, 1995) provide details on the synthesis, labeling, detection and quantification of DNA and RNA, including oligonucleotides.

“Stringent hybridization wash conditions” in the context of nucleic acid hybridization experiments, such as Southern and northern hybridizations, are sequence dependent, and are different under different environmental parameters. An extensive guide to the hybridization of nucleic acids is found in Tijssen (1993), supra, and in Hames and Higgins 1 and Hames and Higgins 2, supra.

For purposes of the present invention, generally, “highly stringent” hybridization and wash conditions are selected to be about 5° C. or less lower than the thermal melting point (Tₘ) for the specific sequence at a defined ionic strength and pH (as noted below, highly stringent conditions can also be referred to in comparative terms). The Tₘ is the temperature (under defined ionic strength and pH) at which 50% of the test sequence hybridizes to a perfectly matched primer. Very stringent conditions are selected to be equal to the Tₘ for a particular primer.

The Tₘ is the temperature of the nucleic acid duplexes indicates the temperature at which the duplex is 50% denatured under the given conditions and its represents a direct measure of the stability of the nucleic acid hybrid. Thus, the Tₘ corresponds to the temperature corresponding to the midpoint in transition from helix to random coil; it depends on length, nucleotide composition, and ionic strength for long stretches of nucleotides.

After hybridization, unhybridized nucleic acid material can be removed by a series of washes, the stringency of which can be adjusted depending upon the desired results. Low stringency washing conditions (e.g., using higher salt and lower temperature) increase sensitivity, but can product nonspecific hybridization signals and high background signals. Higher stringency conditions (e.g., using lower salt and higher temperature that is closer to the hybridization temperature) lowers the background signal, typically with only the specific signal remaining. See, Molecular Biomethods Handbook (Rapley and Walker Eds., Humana Press, Inc., 1998) (hereinafter “Rapley and Walker”), which is incorporated herein by reference in its entirety for all purposes.

Thus, one measure of stringent hybridization is the ability of the primer to hybridize to one or more of the target nucleic acids (or complementary polynucleotide sequences thereof) under highly stringent conditions. Stringent hybridization and wash conditions can easily be determined empirically for any test nucleic acid.

For example, in determining highly stringent hybridization and wash conditions, the hybridization and wash conditions are gradually increased (e.g., by increasing temperature, decreasing salt concentration, increasing detergent concentration and/or increasing the concentration of organic solvents, such as formalin, in the hybridization or wash), until a selected set of criteria are met. For example, the hybridization and wash conditions are gradually increased until a target nucleic acid, and complementary polynucleotide sequences thereof, binds to a perfectly matched complementary nucleic acid.

A target nucleic acid is said to specifically hybridize to a primer nucleic acid when it hybridizes at least half as well to the primer as to a perfectly matched complementary target, i.e., with a signal to noise ratio at least half as high as hybridization of the primer to the target under conditions in which the perfectly matched primer binds to the perfectly matched complementary target with a signal to noise ratio that is at least about 2.5 times to 10 times, typically 5 times to 10 times as high as that observed for hybridization to any of the unmatched target nucleic acids.

Optionally, primers are designed such that the annealing temperature of the universal sequence is higher/greater than that of the target-specific sequences. Method employing these primers further include increasing the annealing temperature of the reaction after the first few rounds of amplification. This increase in reaction temperature suppresses further amplification of sample nucleic acids by the TSPs, and drives amplification by the UP. Depending on the application envisioned, one skilled in the art can employ varying conditions of hybridization to achieve varying degrees of selectivity of primer towards the target sequence. For example, varying the stringency of hybridization or the position of primer hybridization can reveal divergence within gene families.

Optionally, each candidate primer is shown or proven to be compatible with the other primers used in a multiplex reaction. In a preferred embodiment, each target-specific primer pair produces a single amplification product of a predicted size from a sample minimally containing all of the targets of the multiplex, and more preferably from a crude RNA mixture. Preferably, amplification of each individual target by its corresponding primers is not inhibited by inclusion of any other primers in the multiplex. None of the primers, either individually or in combination, should produce spurious products. These issues are easily addressed by one of skill in the art without the need for excessive undue experimentation.

Inherent Properties and Labels Primer sequences are optionally designed to accommodate one or more detection techniques that can be employed while performing the methods of the present invention. For example, detection of the amplification products is optionally based upon one or more inherent properties of the amplification products themselves, such as mass or mobility. Other embodiments utilize methods of detection based on monitoring a label associated with the PCR products. In these embodiments, generally one or more of the universal primers contains the label. Optionally, the label is a fluorescent chromaphore. A fluorescent label may be covalently attached, noncovalently intercalated, or may be an energy transfer label. Other useful labels include mass labels, which are incorporated into amplification products and released after the reaction for detection, chemiluminescent labels, electrochemical and infrared labels, isotopic derivatives, nanocrystals, or any of various enzyme-linked or substrate-linked labels detected by the appropriate enzymatic reaction.

One preferred embodiment of the methods of the present invention includes the use and detection of one or more fluorescent labels. Generally, fluorescent molecules each display a distinct emission spectrum, thereby allowing one to employ a plurality of fluorescent labels in a multiplexed reaction, and then separate the mixed data into its component signals by spectral deconvolution. Exemplary fluorescent labels for use in the methods of the present invention include a single dye covalently attached to the molecule being detected, a single dye noncovalently intercalated into product DNA, or an energy-transfer fluorescent label.

Other embodiments of labeling include mass labels, which are incorporated into amplification products and released after the reaction for detection; chemiluminescent, electrochemical, and infrared labels; radioactive isotopes; and any of various enzyme-linked or substrate-linked labels detectable by the appropriate enzymatic reaction. Many other useful labels are known in the art, and one skilled in the art can envision additional strategies for labeling amplification products of the present invention.

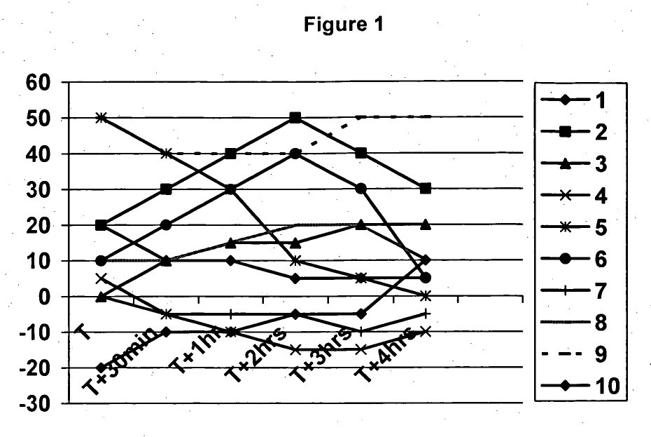

Exemplary Primer Designs for Use in a Multiplexed Amplification Reaction A preferred embodiment of the invention utilizes a combination of TSPs that will hybridize with one of a plurality of designated target sequences, and universal primers (UPs) for amplification of multiple targets in the multiplexed reaction. Optionally, the primary way of separating the signals of the multiplexed amplification is according to product sizes. Alternatively, the signals can be resolved using differential labeling to separate signals from products of similar size. To separate products according to size, the predicted sizes must be considered in primer design. FIG. 1 illustrates the elements of design of these primers. Each of the TSPs has a universal sequence within the 5′ region, which is shared among the primers, but not contained in the original template (i.e. the target sequence). This universal sequence may be the same or different for the forward and reverse TSPs. Following the 3′ end of the universal sequence is a target-specific sequence for annealing to and amplifying the target sequence (e.g., gene) of interest.

The universal primer is composed of the universal sequence held in common within the 5′ regions of the TSPs. If a single UP is to be used, the universal sequence will be the same within all TSPs. If a UP pair is to be used, the universal sequence will be different in the forward and reverse primers of the TSPs. The UP may also contain a detectable label on at least one of the primers, such as a fluorescent chromaphore. Both the target-specific and universal sequences are of sufficient length and sequence complexity to form stable and specific duplexes, allowing amplification and detection of the target gene.

Elimination of Variations in Primer Annealing Efficiency Variations in primer length and sequence can also have a large impact on the efficiency with which primers anneal to their target and prime replication. In a typical multiplexed reaction in which each product is amplified by a unique primer pair, the relative quantities of amplified products may be significantly altered from the relative quantities of targets due to difference in annealing efficiencies. Embodiments of the methods of the present invention that couple the use of target-specific primers and universal primers eliminates this bias, producing amplification products that accurately reflect relative mRNA levels.

Coupled Target-Specific and Universal Priming of the PCR In the methods of the present invention, the amounts of each designated target are amplified to improve the sensitivity and dynamic range of the assay. In some embodiments to monitor gene expression, cellular RNA is isolated and reverse transcribed to obtain cDNA, which is then used as template for amplification. In other embodiments, cDNA may be provided and used directly. The primers described for use in the present invention can be used in any one of a number of template-dependent processes that amplify sequences of the target gene and/or its expressed transcripts present in a given sample. Other types of templates may also be used, such as tRNA, rRNA, or other transcription products, genomic DNA, viral nucleic acids, and synthetic nucleic acid polymers. Several methods described below are contemplated.

A preferred embodiment of the methods of the present invention employs PCR, which is described in detail in U.S. Pat. No. 4,683,195 (Mullis et al.), U.S. Pat. No. 4,683,202 (Mullis), and U.S. Pat. No. 4,800,159 (Mullis et al.), and in PCR Protocols A Guide to Methods and Applications (Innis et al., Eds., Academic Press Inc. San Diego, Calif., 1990). PCR utilizes pairs of primers having sequences complimentary to opposite strands of target nucleic acids, and positioned such that the primers are converging. The primers are incubated with template DNA under conditions that permit selective hybridization. Primers may be provided in double-stranded or single-stranded form, although the single-stranded form is preferred. If the target gene(s) sequence is present in a sample, the primers will hybridize to form a nucleic-acid: primer complex. An excess of deoxynucleoside triphosphates is added, along with a thermostable DNA polymerase, e.g. Taq polymerase. If the target gene(s):primer complex has been formed, the polymerase will extend the primer along the target gene(s) sequence by adding nucleotides. After polymerization, the newly-synthesized strand of DNA is dissociated from its complimentary template strand by raising the temperature of the reaction mixture. When the temperature is subsequently lowered, new primers will bind to each of these two strands of DNA, and the process is repeated. Multiple cycles of raising and lowering the temperature are conducted, with a round of replication in each cycle, until a sufficient amount of amplification product is produced.

In early rounds of the amplification, replication is primed primarily by the TSPs. The first round will add the universal sequence to the 5′ regions of the amplification products. The second cycle will generate sequence complementary to the universal sequence within the 3′ region of the complementary strand, creating a template that can be amplified by the universal primers alone. Optionally, the reaction is designed to contain limiting amounts of each of the TSPs and a molar excess of the UP, such that the UP will generally prime replication once its complementary sequence has been established in the template. The molar excess of UP over a TSP can range from about 5:1 to about 100:1; optionally, the reaction utilizes approximately 10:1 molar excess of UP over the amount of each TSP. Because all of the TSPs contain the same universal sequence, the same universal primer will amplify all targets in the multiplex, eliminating the quantitative variation that results from amplification from different primers.

Amplification Methods In a preferred embodiment of the methods of the present invention, RNA is converted to cDNA using a target-specific primer complementary to the RNA for each gene target being monitored in the multiplex set in a reverse-transcription (RT) reaction. Methods of reverse transcribing RNA into cDNA are well known, and described in Sambrook, supra. Alternative methods for reverse transcription utilize thermostable DNA polymerases, as described in the art. As an exemplary embodiment, avian myeloblastosis virus reverse transcriptase (AMV-RT), or Maloney murine leukemia virus reverse transcriptase (MoMLV-RT) is used, although other enzymes are contemplated. An advantage of using target-specific primers in the RT reaction is that only the desired sequences are converted into a PCR template. No superfluous primers or cDNA products are carried into the subsequent PCR amplification.

In another embodiment of the amplifying step, RNA targets are reverse transcribed using non-specific primers, such as an anchored oligo-dT primer, or random sequence primers. An advantage of this embodiment is that the “unfractionated” quality of the mRNA sample is maintained because the sites of priming are non-specific, i.e., the products of this RT reaction will serve as template for any desired target in the subsequent PCR amplification. This allows samples to be archived in the form of DNA, which is more stable than RNA.

In other embodiments of the methods of the present invention, transcription-based amplification systems (TAS) are used, such as that first described by Kwoh et al. (86(4) Proc. NATL. ACAD. SCI. 1173-1177 (1989)), or isothermal transcription-based systems such as 3SR (Self-Sustained Sequence Replication; Guatelli et al. (87 PROC. NATL. ACAD. SCI. USA, 1874-1878 (1990)) or NASBA (nucleic acid sequence based amplification; Kievits et al. (35(3) J VIROL METHODS. 273-86 (1991)). In these methods, the mRNA target of interest is copied into cDNA by a reverse transcriptase. The primer for cDNA synthesis includes the promoter sequence of a designated DNA-dependent RNA polymerase 5‘to the primer’s region of homology with the template. The resulting cDNA products can then serve as templates for multiple rounds of transcription by the appropriate RNA polymerase. Transcription of the cDNA template rapidly amplifies the signal from the original target mRNA. The isothermal reactions bypass the need for denaturing cDNA strands from their RNA templates by including RNAse H to degrade RNA hybridized to DNA.

In other embodiments, amplification is accomplished by used of the ligase chain reaction (LCR), disclosed in European Patent Application No. 320,308 (Backman and Wang), or by the ligase detection reaction (LDR), disclosed in U.S. Pat. No. 4,883,750 (Whiteley et al.). In LCR, two probe pairs are prepared, which are complimentary each other, and to adjacent sequences on both strands of the target. Each pair will bind to opposite strands of the target such that they abut. Each of the two probe pairs can then be linked to form a single unit, using a thermostable ligase. By temperature cycling, as in PCR, bound ligated units dissociate from the target, then both molecules can serve as “target sequences” for ligation of excess probe pairs, providing for an exponential amplification. The LDR is very similar to LCR. In this variation, oligonucleotides complimentary to only one strand of the target are used, resulting in a linear amplification of ligation products, since only the original target DNA can serve as a hybridization template. It is used following a PCR amplification of the target in order to increase signal.

In further embodiments, several methods generally known in the art would be suitable methods of amplification. Some additional examples include, but are not limited to, strand displacement amplification (Walker et al, 20 NUCLEIC ACIDS RES. 1691-1696 (1992)), repair chain reaction (REF), cyclic probe reaction (REF), solid-phase amplification, including bridge amplification (Mehta and Singh, 26(6) BIOTECHNIQUES 1082-1086 (1999)), rolling circle amplification (Kool, U.S. Pat. No. 5,714,320), rapid amplification of cDNA ends (Frohman, 85 PROC. NATL. ACAD. SCI. 8998-9002 (1988)), and the “invader assay” (Griffin et al., 96 PROC. NATL. ACAD. SCI. 6301-6306 (1999).

Attenuation of Strong Signals The set of targets included in a multiplex reaction generally all yield signal strengths within the dynamic range of the detection platform used in order for quantitation of gene expression to be accurate. In some embodiments, it may be desirable or necessary to include a very highly expressed gene in a multiplex assay. However, the highly-expressed gene can impact the accuracy of quantitation for other genes expressed at very low levels if its signal is not attenuated. The methods of the current invention provide ways for attenuating the signals of relatively abundant targets during the amplification reaction such that they can be included in a multiplexed set without impacting the accuracy of quantitation of that set.

Toward this end, amplification primers are optionally used that block polymerase extension of the 3′ end of the primer. One preferred embodiment is modification of the 3′-hydroxyl of the oligonucleotide primer by addition of a phosphate group. Another preferred embodiment is attachment of the terminal nucleotide via a 3′-3′ linkage. One skilled in the art can conceive of other chemical structures or modifications that can be used for this purpose. The modified and the corresponding unmodified primer for the highly abundant target are mixed in a ratio empirically determined to reduce that target's signal, such that it falls within the dynamic range of other targets of the multiplex. Preferably, the reverse target-specific primer is modified, thereby attenuating signal by reduction of the amount of template created in the reverse transcriptase reaction.

Another embodiment for signal attenuation entails use of a target-specific primer that contains the target-specific sequence, but no universal primer sequence. This abbreviated primer (sans universal sequence) and the corresponding primer containing the universal sequence within the 5′ region are mixed in a ratio empirically determined to reduce that target's signal, such that it then falls within the dynamic range of other targets of the multiplex system.

Data Collection The number of species than can be detected within a mixture depends primarily on the resolution capabilities of the separation platform used, and the detection methodology employed. A preferred embodiment of the separation step of the methods of the present invention is based upon size-based separation technologies. Once separated, individual species are detected and quantitated by either inherent physical characteristics of the molecules themselves, or detection of a label associated with the DNA.

Embodiments employing other separation methods are also described. For example, certain types of labels allow resolution of two species of the same mass through deconvolution of the data. Non-size based differentiation methods (such as deconvolution of data from overlapping signals generated by two different fluorophores) allow pooling of a plurality of multiplexed reactions to further increase throughput.

Optionally, the throughput rate for the detection step is between about 100 and 5000 samples per hour, preferably between about 250 and 2500 samples, and more preferably about 1000 samples per hour per separation system (i.e., one mass spectrometer, one lane of a gel, or one capillary of a capillary electrophoresis device). In order to further reduce assay costs and increase the throughput of the overall process, sample-handling is optionally conducted in a miniaturized format. For the methods of the present invention, miniaturized formats are those conducted at submicroliter volumes, including both microfluidic and nanofluidic platforms. Any or all of the amplification, separation, and/or detection steps of the present can utilize miniaturized formats and platforms. For example, many of the modes of separation described below are presently available in a miniaturized scale.

Separation Methods Preferred embodiments of the present invention incorporate a step of separating the products of a reaction based on their size differences. The PCR products generated during the multiplex amplification optionally range from about 50 to about 500 bases in length, which can be resolve from one another by size. Any one of several devices may be used for size separation, including mass spectrometry, any of several electrophoretic devices, including capillary, polyacrylamide gel, or agarose gel electrophoresis, or any of several chromatographic devices, including column chromatography, HPLC, or FPLC.

One preferred embodiment for sample analysis is mass spectrometry. Several modes of separation that determine mass are possible, including Time-of-Flight (TOF), Fourier Transform Mass Spectrometry (FFMS), and quadruple mass spectrometry. Possible methods of ionization include Matrix-Assisted Laser Desorption and Ionization (MALDI) or Electrospray Ionization (ESI). A preferred embodiment for the uses described in this invention is MALDI-TOF (Wu et al., 7 RAPID COMMUNICATIONS IN MASS SPECTROMETRY 142-146 (1993)). This method may be used to provide unfragmented mass spectra of mixed-base oligonucleotides containing between about 1 and about 1000 bases. In preparing the sample for analysis, the analyte is mixed into a matrix of molecules that resonantly absorb light at a specified wavelength. Pulsed laser light is then used to desorb oligonucleotide molecules out of the absorbing solid matrix, creating free, charged oligomers and minimizing fragmentation. The preferred solid matrix material for this purpose is 3-hydroxypicolinic acid (Wu, supra), although others are contemplated.

In another preferred embodiment, the device of the invention is a microcapillary for analysis of nucleic acids obtained from the sample. Microcapillary electrophoresis generally involves the use of a thin capillary or channel, which may optionally be filled with a particular medium to improve separation, and employs an electric field to separate components of the mixture as the sample travels through the capillary. Samples composed of linear polymers of a fixed charge-to-mass ratio, such as DNA, will separate based on size. The high surface to volume ratio of these capillaries allows application of very high electric fields across the capillary without substantial thermal variation, consequently allowing very rapid separations. When combined with confocal imaging methods, these methods provide sensitivity in the range of attomoles, comparable to the sensitivity of radioactive sequencing methods. The use of microcapillary electrophoresis in size separation of nucleic acids has been reported in Woolley and Mathies (91 PROC. NATL. ACAD. SCI. USA 11348-11352 (1994)).

Capillaries are optionally fabricated from fused silica, or etched, machined, or molded into planar substrates. In many microcapillary electrophoresis methods, the capillaries are filled with an appropriate separation/sieving matrix. Several sieving matrices are known in the art that may be used for this application, including, e.g., hydroxyethyl cellulose, polyacrylamide, agarose, and the like. Generally, the specific gel matrix, running buffers and running conditions are selected to obtain the separation required for a particular application. Factors that are considered include, e.g., sizes of the nucleic acid fragments, level of resolution, or the presence of undenatured nucleic acid molecules. For example, running buffers may include agents such as urea to denature double-stranded nucleic acids in a sample.

Microfluidic systems for separating molecules such as DNA and RNA are commercially available and are optionally employed in the methods of the present invention. For example, the “Personal Laboratory System” and the “High Throughput System” have been developed by Caliper Technologies, Corp. (Mountain View, Calif.). The Agilent 2100, which uses Caliper Technologies' LabChip™ microfluidic systems, is available from Agilent Technologies (Palo Alto, Calif.). Currently, specialized microfluidic devices which provide for rapid separation and analysis of both DNA and RNA are available from Caliper Technologies for the Agilent 2100.

Other embodiments are generally known in the art for separating PCR amplification products by electrophoresis through gel matrices. Examples include polyacrylamide, agarose-acrylamide, or agarose gel electrophoresis, using standard methods (Sambrook, supra).

Alternatively, chromatographic techniques may be employed for resolving amplification products. Many types of physical or chemical characteristics may be used to effect chromatographic separation in the present invention, including adsorption, partitioning (such as reverse phase), ion-exchange, and size exclusion. Many specialized techniques have been developed for their application including methods utilizing liquid chromatography or HPLC (Katz and Dong, 8(5) BIOTECHNIQUES 546-55 (1990); Gaus et al., 158 J. IMMUNOL. METHODS 229-236 (1993)).

In yet another embodiment of the separation step of the present invention, cDNA products are captured by their affinity for certain substrates, or other incorporated binding properties. For example, labeled cDNA products such as biotin or antigen can be captured with beads bearing avidin or antibody, respectively. Affinity capture is utilized on a solid support to enable physical separation. Many types of solid supports are known in the art that would be applicable to the present invention. Examples include beads (e.g. solid, porous, magnetic), surfaces (e.g. plates, dishes, wells, flasks, dipsticks, membranes), or chromatographic materials (e.g. fibers, gels, screens).

Certain separation embodiments entail the use of microfluidic techniques. Technologies include separation on a microcapillary platform, such as designed by ACLARA BioSciences Inc. (Mountain View, Calif.), or the LabChip™ microfluidic devices made by Caliper Technologies Inc. Another recent technology developed by Nanogen, Inc. (San Diego, Calif.), utilizes microelectronics to move and concentrate biological molecules on a semiconductor microchip. The microfluidics platforms developed at Orchid Biosciences, Inc. (Princeton, N.J.), including the Chemtel™ Chip which provides for parallel processing of hundreds of reactions, can be used in the present invention. These microfluidic platforms require only nanoliter sample volumes, in contrast to the microliter volumes required by other conventional separation technologies.

Fabrication of microfluidic devices, including microcapillary electrophoretic devices, has been discussed in detail, e.g., Regnier et al. (17(3) TRENDS BIOTECHNOL. 101-106 (1999)), Deyl et al. (92 FORENSIC SCI. INT. 89-124 (1998)), Effenhauser et al. (18 ELECTROPHORESIS 2203-2213 (1997)), and U.S. Pat. No. 5,904,824 (Oh). Typically, the methods make use of photolithographic etching of micron-scale channels on a silica, silicon, or other crystalline substrate or chip. In some embodiments, capillary arrays may be fabricated using polymeric materials with injection-molding techniques. These methods can be readily adapted for use in miniaturized devices of the present invention.

Some of the processes usually involved in genetic analysis have been miniaturized using microfluidic devices. For example, PCT publication WO 94/05414 reports an integrated micro-PCR apparatus for collection and amplification of nucleic acids from a specimen. U.S. Pat. No. 5,304,487 (Wilding et al.) and U.S. Pat. No. 5,296,375 (Kricka et al.) discuss devices for collection and analysis of cell-containing samples. U.S. Pat. No. 5,856,174 (Lipshutz et al.) describes an apparatus that combines the various processing and analytical operations involved in nucleic acid analysis.

Additional technologies are also contemplated. For example, Kasianowicz et al. (98 PROC. NATL. ACAD. SCI. USA 13770-13773 (1996)) describe the use of ion channel pores in a lipid bilayer membrane for determining the length of polynucleotides. In this system, an electric field is generated by the passage of ions through the pores. Polynucleotide lengths are measured as a transient decrease of ionic current due to blockage of ions passing through the pores by the nucleic acid. The duration of the current decrease was shown to be proportional to polymer length. Such a system can be applied as a size separation platform in the present invention.

The target-specific primers and universal primers of the present invention are useful both as reagents for hybridization in solution, such as priming PCR amplification, as well as for embodiments employing a solid phase, such as microarrays. With microarrays, sample nucleic acids such as mRNA or DNA are fixed on a selected matrix or surface. PCR products may be attached to the solid surface via one of the amplification primers, then denatured to provide single-stranded DNA. This-spatially-partitioned, single-stranded nucleic acid is then subject to hybridization with selected probes under conditions that allow a quantitative determination of target abundance. In this embodiment, amplification products from each individual multiplexed reaction are not physically separated, but are differentiated by hybridizing with a set of probes that are differentially labeled. Alternatively, unextended amplification primers may be physically immobilized at discreet positions on the solid support, then hybridized with the products of a multiplexed PCR amplification for quantitation of distinct species within the sample. In this embodiment, amplification products are separated by way of hybridization with probes that are spatially separated on the solid support.

Separation platforms may optionally be coupled to utilize two different separation methodologies, thereby increasing the multiplexing capacity of reactions beyond that which can be obtained by separation in a single dimension. For example, some of the RT-PCR primers of a multiplex reaction may be coupled with a moiety that allows affinity capture, while other primers remain unmodified. Samples are then passed through an affinity chromatography column to separate PCR products arising from these two classes of primers. Flow-through fractions are collected and the bound fraction eluted. Each fraction may then be further separated based on other criteria, such as size, to identify individual components.

The invention also includes rapid analytical method using one or more microfluidic handling systems. For example, a subset of primers in a multiplex reaction would contain a hydrophobic group. Separation is then performed in two dimensions, with hydrophilic partitioning in one direction, followed by size separation in the second direction. The use of a combination of dyes can further increase the multiplex size.

Detection Methods Following separation of the different products of the multiplex, one or more of the member species is detected and/or quantitated. Some embodiments of the methods of the present invention enable direct detection of products. Other embodiments detect reaction products via a label associated with one or more of the amplification primers. Many types of labels suitable for use in the present invention are known in the art, including chemiluminescent, isotopic, fluorescent, electrochemical, inferred, or mass labels, or enzyme tags. In further embodiments, separation and detection may be a multi-step process in which samples are fractionated according to more than one property of the products, and detected one or more stages during the separation process.

One embodiment of the invention requiring no labeling or modification of the molecules being analyzed is detection of the mass-to-charge ratio of the molecule itself. This detection technique is optionally used when the separation platform is a mass spectrometer. An embodiment for increasing resolution and throughput with mass detection is in mass-modifying the amplification products. Nucleic acids can be mass-modified through either the amplification primer or the chain-elongating nucleoside triphosphates. Alternatively, the product mass can be shifted without modification of the individual nucleic acid components, by instead varying the number of bases in the primers. Several types of moieties have been shown to be compatible with analysis by mass spectrometry, including polyethylene glycol, halogens, alkyl, aryl, or aralkyl moieties, peptides (described in, for example, U.S. Pat. No. 5,691,141). Isotopic variants of specified atoms, such as radioisotopes or stable, higher mass isotopes, are also used to vary the mass of the amplification product. Radioisotopes can be detected based on the energy released when they decay, and numerous applications of their use are generally known in the art. Stable (non-decaying) heavy isotopes can be detected based on the resulting shift in mass, and are useful for distinguishing between two amplification products that would otherwise have similar or equal masses. Other embodiments of detection that make use of inherent properties of the molecule being analyzed include ultraviolet light absorption (UV) or electrochemical detection. Electrochemical detection is based on oxidation or reduction of a chemical compound to which a voltage has been applied. Electrons are either donated (oxidation) or accepted (reduction), which can be monitored as current. For both UV absorption and electrochemical detection, sensitivity for each individual nucleotide varies depending on the component base, but with molecules of sufficient length this bias is insignificant, and detection levels can be taken as a direct reflection of overall nucleic acid content.

Several embodiments of the detecting step of the present invention are designed to identify molecules indirectly by detection of an associated label. A number of labels may be employed that provide a fluorescent signal for detection, such as those commercially available from Invitrogen Corp. (Carlsbad, Calif.). If a sufficient quantity of a given species is generated in a reaction, and the mode of detection has sufficient sensitivity, then some fluorescent molecules may be incorporated into one or more of the primers used for amplification, generating a signal strength proportional to the concentration of DNA molecules. Several fluorescent moieties, including Alexa 350, Alexa 430, AMCA, BODIPY 630/650, BODIPY 650/665, BODIPY-FL, BODIPY-R6G, BODIPY-TMR, BODIPY-TRX, carboxyfluorescein, Cascade Blue, Cy3, Cy5, 6-FAM, Fluorescein, HEX, 6-JOE, Oregon Green 488, Oregon Green 500, Oregon Green 514, Pacific Blue, REG, Rhodamine Green, Rhodarmine Red, ROX, TAMRA, TET, Tetramethylrhodamine, and Texas Red, are generally known in the art and routinely used for identification of discreet nucleic acid species, such as in sequencing reactions. Many of these dyes have emission spectra distinct from one another, enabling deconvolution of data from incompletely resolved samples into individual signals. This allows pooling of separate reactions that are each labeled with a different dye, increasing the throughput during analysis, as described in more detail below.

Several embodiments of the detecting step of the present invention are designed to identify molecules indirectly by detection of an associated label. A number of labels may be employed that provide a fluorescent signal for detection (see, for example, www.probes.com). If a sufficient quantity of a given species is generated in a reaction, and the mode of detection has sufficient sensitivity, then some fluorescent molecules may be incorporated into one or more of the primers used for amplification, generating a signal strength proportional to the concentration of DNA molecules. Several fluorescent moieties, including Alexa 350, Alexa 430, AMCA, BODIPY 630/650, BODIPY 650/665, BODIPY-FL, BODIPY-R6G, BODIPY-TMR, BODIPY-TRX, carboxyfluorescein, Cascade Blue, Cy3, Cy5, 6-FAM, Fluorescein, HEX, 6-JOE, Oregon Green 488, Oregon Green 500, Oregon Green 514, Pacific Blue, REG, Rhodamine Green, Rhodarmine Red, ROX, TAMRA, TET, Tetramethylrhodamine, and Texas Red, are generally known in the art and routinely used for identification of discreet nucleic acid species, such as in sequencing reactions. Many of these dyes have emission spectra distinct from one another, enabling deconvolution of data from incompletely resolved samples into individual signals. This allows pooling of separate reactions that are each labeled with a different dye, increasing the throughput during analysis, as described in more detail below.

The signal strength obtained from fluorescent dyes can be enhanced through use of related compounds called energy transfer (ET) fluorescent dyes. After absorbing light, ET dyes have emission spectra that allow them to serve as “donors” to a secondary “acceptor” dye that will absorb the emitted light and emit a lower energy fluorescent signal. Use of these coupled-dye systems can significantly amplify fluorescent signal. Examples of ET dyes include the ABI PRISM BigDye terminators, recently commercialized by Perkin-Elmer Corporation (Foster City, Calif.) for applications in nucleic acid analysis. These chromaphores incorporate the donor and acceptor dyes into a single molecule and an energy transfer linker couples a donor fluorescein to a dichlororhodamine acceptor dye, and the complex is attached to a DNA replication primer.

Fluorescent signals can also be generated by non-covalent intercalation of fluorescent dyes into nucleic acids after their synthesis and prior to separation. This type of signal will vary in intensity as a function of the length of the species being detected, and thus signal intensities must be normalized based on size. Several applicable dyes are known in the art, including, but not limited to, ethidium bromide and Vistra Green. Some intercalating dyes, such as YOYO or TOTO, bind so strongly that separate DNA molecules can each be bound with a different dye and then pooled, and the dyes will not exchange between DNA species. This enables mixing separately generated reactions in order to increase multiplexing during analysis.

Alternatively, technologies such as the use of nanocrystals as a fluorescent DNA label (Alivisatos et al., 382 NATURE 609-11 (1996)) can be employed in the methods of the present invention. Another method, described by Mazumder et al. (26 NUCLEIC ACIDS RES. 1996-2000 (1998)), describes hybridization of a labeled oligonucleotide probe to its target without physical separation from unhybridized probe. In this method, the probe is labeled with a chemiluminescent molecule that in the unbound form is destroyed by sodium sulfite treatment, but is protected in probes that have hybridized to target sequence.

In another embodiment, products may be detected and quantitated by monitoring a set of mass labels, each of which are specifically associated with one species of amplification reaction. The labels are released by either chemical or enzymatic mechanisms after the amplification reaction. Release is followed by size separation of the mixture of labels to quantitate the amount of each species of the amplification reaction. Separation methods that can be employed include mass spectrometry, capillary electrophoresis, or HPLC. Such strategies, and their applications for detection of nucleic acids, have been described in, for example, U.S. Pat. No. 6,104,028 (Hunter et al.) and U.S. Pat. No. 6,051,378 (Monforte et al.), as well as PCT publications WO 98/26095 (Monforte et al.) and WO 97/27327 (Van Ness et al.).

In further embodiments, both electrochemical and infrared methods of detection can be amplified over the levels inherent to nucleic acid molecules through attachment of EC or IR labels. Their characteristics and use as labels are described in, for example, PCT publication WO 97/27327. Some preferred compounds that can serve as an IR label include an aromatic nitrile, aromatic alkynes, or aromatic azides. Numerous compounds can serve as an EC label; many are listed in PCT publication WO 97/27327.

Enzyme-linked reactions are also employed in the detecting step of the methods of the present invention. Enzyme-linked reactions theoretically yield an infinite signal, due to amplification of the signal by enzymatic activity. In this embodiment, an enzyme is linked to a secondary group that has a strong binding affinity to the molecule of interest. Following separation of the nucleic acid products, enzyme is bound via this affinity interaction. Nucleic acids are then detected by a chemical reaction catalyzed by the associated enzyme. Various coupling strategies are possible utilizing well-characterized interactions generally known in the art, such as those between biotin and avidin, an antibody and antigen, or a sugar and lectin. Various types of enzymes can be employed, generating colorimetric, fluorescent, chemiluminescent, phosphorescent, or other types of signals. As an illustration, a PCR primer may be synthesized containing a biotin molecule. After PCR amplification, DNA products are separated by size, and those made with the biotinylated primer are detected by binding with streptavidin that is covalently coupled to an enzyme, such as alkaline phosphatase. A subsequent chemical reaction is conducted, detecting bound enzyme by monitoring the reaction product. The secondary affinity group may also be coupled to an enzymatic substrate, which is detected by incubation with unbound enzyme. One of skill in the art can conceive of many possible variations on the different embodiments of detection methods described above.

In some embodiments, it may be desirable prior to detection to separate a subset of amplification products from other components in the reaction, including other products. Exploitation of known high-affinity biological interactions can provide a mechanism for physical capture. In some embodiments of this process, the 5′ region of one of the universal primers contains a binding moiety that allows capture of the products of that primer. Some examples of high-affinity interactions include those between a hormone with its receptor, a sugar with a lectin, avidin and biotin, or an antigen with its antibody. After affinity capture, molecules are retrieved by cleavage, denaturation, or eluting with a competitor for binding, and then detected as usual by monitoring an associated label. In some embodiments, the binding interaction providing for capture may also serve as the mechanism of detection.

Furthermore, the size of an amplification product or products are optionally changed, or “shifted,” in order to better resolve the amplification products from other products prior to detection. For example, chemically cleavable primers can be used in the amplification reaction. In this embodiment, one or more of the primers used in amplification contains a chemical linkage that can be broken, generating two separate fragments from the primer. Cleavage is performed after the amplification reaction, removing a fixed number of nucleotides from the 5′ end of products made from that primer. Design and use of such primers is described in detail in, for example, PCT publication WO 96/37630.

One preferred embodiment of the methods of the present invention is the generation of gene expression profiles. However, several other applications are also possible, as would be apparent to one skilled in the art from a reading of this disclosure. For example, the methods of the present invention can be used to investigate the profile and expression levels of one or more members of complex gene families. As an illustration, cytochrome P-450 isozymes form a complex set of closely related enzymes that are involved in detoxification of foreign substances in the liver. The various isozymes in this family have been shown to be specific for different substrates. Design of target-specific primers that anneal to variant regions in the genes provides an assay by which their relative levels of induction in response to drug treatments can be monitored. Other examples include monitoring expression levels of alleles with allele-specific primers, or monitoring mRNA processing with primers that specifically hybridize to a spliced or unspliced region, or to splice variants. One skilled in the art could envision other applications of the present invention that would provide a method to monitor genetic variations or expression mechanisms.

Systems for Gene Expression Analysis The present invention also provides systems for analyzing gene expression. The elements of the system include, but are not limited to, an amplification module for producing a plurality of amplification products from a pool of target sequences; a detection module for detecting one or more members of the plurality of amplification products and generating a set of gene expression data; and an analyzing module for organizing and/or analyzing the data points in the data set. Any or all of these modules can comprise high throughput technologies and/or systems.

The amplification module of the system of the present invention produces a plurality of amplification products from a pool of target sequences. The amplification module includes at least one pair of universal primers and at least one pair of target-specific primers for use in the amplification process. Optionally, the amplification module includes a unique pair of universal primers for each target sequence. Furthermore, the amplification module can include components to perform one or more of the following reactions: a polymerase chain reaction, a transcription-based amplification, a self-sustained sequence replication, a nucleic acid sequence based amplification, a ligase chain reaction, a ligase detection reaction, a strand displacement amplification, a repair chain reaction, a cyclic probe reaction, a rapid amplification of cDNA ends, an invader assay, a bridge amplification, a rolling circle amplification, solution phase and/or solid phase amplifications, and the like.

The detection module detects the presence, absence, or quantity of one or more members of the plurality of amplification products. Additionally, the detection module generates a set of gene expression data, generally in the form of a plurality of data points. The detection module optionally further comprises a separation module for separation of one or more members of the multiplexed reaction prior to, or during, operation of the detection module. The detection module, or the optional separation module, can include systems for implementing separation of the amplification products; exemplary detection modules include, but are not limited to, mass spectrometry instrumentation and electrophoretic devices.

The third component of the system of the present invention, the analyzing module, is in operational communication with the detection module. The analyzing module of the system includes, e.g., a computer or computer-readable medium having one or more one or more logical instructions for analyzing the plurality of data points generated by the detection system. The analyzing system optionally comprises multiple logical instructions; for example, the logical instructions can include one or more instructions which organize the plurality of data points into a database and one or more instructions which analyze the plurality of data points. The instructions can include software for performing difference analysis upon the plurality of data points. Additionally (or alternatively), the instructions can include or be embodied in software for generating a graphical representation of the plurality of data points. Optionally, the instructions can be embodied in system software which performs combinatorial analysis on the plurality of data points.

The computer employed in the analyzing module of the present invention can be, e.g., a PC (Intel x86 or Pentium chip-compatible DOS™, OS2™, WINDOWS™, WINDOWS NT™, WINDOWS95™, WINDOWS98™, or WINDOWS ME™), a LINUX based machine, a MACINTOSH™, Power PC, or a UNIX based machine (e.g., SUN™ work station) or other commercially common computer which is known to one of skill. Software for computational analysis is available, or can easily be constructed by one of skill using a standard programming language such as VisualBasic, Fortran, Basic, C, C++, Java, or the like. Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can also be used in the analyzing system of the present invention.

The computer optionally includes a monitor that is often a cathode ray tube (“CRT”) display, a flat panel display (e.g., active matrix liquid crystal display, liquid crystal display), or others. Computer circuitry is often placed in a box that includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of sequences to be compared or otherwise manipulated in the relevant computer system.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation.

The software can also include output elements for displaying and/or further analyzing raw data, massaged data, or proposed results from one or more computational processes involved in the analysis of the gene expression data set.

Databases Data collected from the subjects may be stored in one or more databases. Any suitable data storage technique or media may be used in the method of the present invention including, but not limited to, electronic data storage media. The database is used as a repository for patent information or for reference purposes to compare with subsequent data collected from the patient or another patient, for example.

Kits In an additional aspect, the present invention provides kits embodying the methods, compositions, and systems for analysis of gene expression as described herein. Kits of the present invention optionally comprise one or more of the following, preferably in a spatially separate arrangement: a) at least one pair of universal primers; b) at least one pair of target-specific primers; c) at least one pair of reference gene-specific primers; and d) one or more amplification reaction enzymes, reagents, or buffers. Optionally, the universal primers provided in the kit include labeled primers, such as those described in the present application and the references cited herein. The target-specific primers can vary from kit to kit, depending upon the specified target gene(s) to be investigated. Exemplary reference gene-specific primers (e.g., target-specific primers for directing transcription of one or more reference genes) include, but are not limited to, primers for .beta.-actin, cyclophilin, GAPDH, and various rRNA molecules.

The kits of the invention optionally include one or more preselected primer sets that are specific for the genes to be amplified. The preselected primer sets optionally comprise one or more labeled nucleic acid primers, contained in suitable receptacles or containers. Exemplary labels include, but are not limited to, a fluorophore, a dye, a radiolabel, an enzyme tag, etc., that is linked to a nucleic acid primer itself.

In one embodiment, kits that are suitable for use in PCR are provided. In PCR kits, target-specific and universal primers are provided which include sequences that have sequences from, and hybridize to spatially distinct regions of one or more target genes. Optionally, pairs of target-specific primers are provided. Generally, the target-specific primers are composed of at least two parts: a universal sequence within the 5′ portion that is complementary to a universal primer sequence, and a sequence within the 3′ portion (and optionally, proximal to the universal sequence) for recognition of a target gene. In some embodiments of the invention, the set of targets monitored in an analysis may be specified by a client for use in a proprietary testing or screening application. In an alternate embodiment, standardized target sets may be developed for general applications, and constitute components of the kits described below. Kits of either of these embodiment can be used to amplify all genes, unknown and/or known, that respond to certain treatments or stimuli.

In addition, one or more materials and/or reagents required for preparing a biological sample for gene expression analysis are optionally included in the kit. Furthermore, optionally included in the kits are one or more enzymes suitable for amplifying nucleic acids, including various polymerases (RT, Taq, etc.), one or more deoxynucleotides, and buffers to provide the necessary reaction mixture for amplification.

In one preferred embodiment of the invention, the kits are employed for analyzing gene expression patterns using mRNA as the starting template. The mRNA template may be presented as either total cellular RNA or isolated mRNA; both types of sample yield comparable results. In other embodiments, the methods and kits described in the present invention allow quantitation of other products of gene expression, including tRNA, rRNA, or other transcription products. In still further embodiments, other types of nucleic acids may serve as template in the assay, including genomic or extragenomic DNA, viral RNA or DNA, or nucleic acid polymers generated by non-replicative or artificial mechanism, including PNA or RNA/DNA copolymers.

Optionally, the kits of the present invention further include software to expedite the generation, analysis and/or storage of data, and to facilitate access to databases. The software includes logical instructions, instructions sets, or suitable computer programs that can be used in the collection, storage and/or analysis of the data. Comparative and relational analysis of the data is possible using the software provided.

The kits optionally comprise distinct containers for each individual reagent and enzyme, as well as for each probe or primer pair. Each component will generally be suitable as aliquoted in its respective container. The container of the kits optionally includes at least one vial, ampule, or test tube. Flasks, bottles and other container mechanisms into which the reagents can be placed and/or aliquoted are also possible. The individual containers of the kit are preferably maintained in close confinement for commercial sale. Suitable larger containers may include injection or blow-molded plastic containers into which the desired vials are retained. Instructions, such as written directions or videotaped demonstrations detailing the use of the kits of the present invention, are optionally provided with the kit.

In a further aspect, the present invention provides for the use of any composition or kit herein, for the practice of any method or assay herein, and/or for the use of any apparatus or kit to practice any assay or method herein.

The terms “disease” or “condition” are commonly recognized in the art and designate the presence of signs and/or symptoms in an individual or patient that are generally recognized as abnormal. Diseases or conditions may be diagnosed and categorized based on pathological changes. Signs may include any objective evidence of a disease such as changes that are evident by physical examination of a patient or the results of diagnostic tests. Symptoms are subjective evidence of disease or a patient's condition, i.e. the patient's perception of an abnormal condition that differs from normal function, sensation, or appearance, which may include, without limitations, physical disabilities, morbidity, pain, and other changes from the normal condition experienced by an individual. Various diseases or conditions include, but are not limited to; those categorized in standard textbooks of medicine including, without limitation, textbooks of nutrition, allopathic, homeopathic, and osteopathic medicine. In certain aspects of this invention, the disease or condition is selected from the group consisting of the types of diseases listed in standard texts such as Harrison's Principles of Internal Medicine, 14^{th} Edition (Fauci et al, Eds., McGraw Hill, 1997), or Robbins Pathologic Basis of Disease, 6^{th} Edition (Cotran et al, Ed. WB Saunders Co., 1998), or the Diagnostic and Statistical Manual of Mental Disorders: DSM-IV, 4^{th} Edition, (American Psychiatric Press, 1994), or other texts described below.

The term “suffering from a disease or condition” means that a person is either presently subject to the signs and symptoms, or is more likely to develop such signs and symptoms than a normal person in the population. Thus, for example, a person suffering from a condition can include a developing fetus, a person subject to a treatment or environmental condition which enhances the likelihood of developing the signs or symptoms of a condition, or a person who is being given or will be given a treatment which increase the likelihood of the person developing a particular condition. For example, tardive dyskinesia is associated with long-term use of anti-psychotics; dyskinesias, paranoid ideation, psychotic episodes and depression have been associated with use of L-dopa in Parkinson's disease; and dizziness, diplopia, ataxia, sedation, impaired mentation, weight gain, and other undesired effects have been described for various anticonvulsant therapies, alopecia and bone marrow suppression are associated with cancer chemotherapeutic regimens, and immunosuppression is associated with agents to limit graft rejection following transplantation. Thus, methods of the present invention which relate to treatments of patients (e.g., methods for selecting a treatment, selecting a patient for a treatment, and methods of treating a disease or condition in a patient) can include primary treatments directed to a presently active disease or condition, secondary treatments which are intended to cause a biological effect relevant to a primary treatment, and prophylactic treatments intended to delay, reduce, or prevent the development of a disease or condition, as well as treatments intended to cause the development of a condition different from that which would have been likely to develop in the absence of the treatment.

The term “intervention” refers to a process that is intended to produce a beneficial change in the condition of a mammal, e.g., a human, often referred to as a patient. A beneficial change can, for example, include one or more of: restoration of function, reduction of symptoms, limitation or retardation of progression of a disease, disorder, or condition or prevention, limitation or retardation of deterioration of a patient's condition, disease or disorder. Such intervention can involve, for example, nutritional modifications, administration of radiation, administration of a drug, surgery, behavioral modifications, and combinations of these, among others.

The term “intervention” includes administration of “drugs” and “candidate therapeutic agents”. A drug is a chemical entity or biological product, or combination of chemical entities or biological products, administered to a person to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, lipoproteins, and modifications and combinations thereof. A biological product is preferably a monoclonal or polyclonal antibody or fragment thereof such as a variable chain fragment; cells; or an agent or product arising from recombinant technology, such as, without limitation, a recombinant protein, recombinant vaccine, or DNA construct developed for therapeutic, e.g., human therapeutic, use. The term may include, without limitation, compounds that are approved for sale as pharmaceutical products by government regulatory agencies (e.g., U.S. Food and Drug Administration (USFDA or FDA), European Medicines Evaluation Agency (EMEA), and a world regulatory body governing the International Conference of Harmonization (ICH) rules and guidelines), compounds that do not require approval by government regulatory agencies, food additives or supplements including compounds commonly characterized as vitamins, natural products, and completely or incompletely characterized mixtures of chemical entities including natural compounds or purified or partially purified natural products. The term “drug” as used herein is synonymous with the terms “medicine”, “pharmaceutical product”, or “product”. Most preferably the drug is approved by a government agency for treatment of a specific disease or condition. The term “candidate therapeutic agent” refers to a drug or compound that is under investigation, either in laboratory or human clinical testing for a specific disease, disorder, or condition.

The biologically active molecule is most commonly a protein that is subsequently modified by reacting with, or combining with, other constituents of the cell. Such modifications may include, without limitation, modification of proteins to form glycoproteins, lipoproteins, and phosphoproteins, or other modifications known in the art. RNA may be modified without limitation by polyadenylation, splicing, capping or export from the nucleus or by covalent or noncovalent interactions with proteins. The term “gene product” refers to any product directly resulting from transcription of a gene.

In the context of this invention, the term “quantifying RNA expression” refers to determining at least a relative level of an expression of one or more genetic messages in a blood sample.

In this regard, “population” refers to a defined group of individuals or a group of individuals with a particular disease or condition or individuals that may be treated with a specific drug identified by, but not limited to geographic, ethnic, race, gender, and/or cultural indices. In most cases a population will preferably encompass at least ten thousand, one hundred thousand, one million, ten million, or more individuals, with the larger numbers being more preferable. In a preferred aspect of this invention, the population refers to individuals with a specific disease or condition that may be treated with a specific drug.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. “Less effective” means that the treatment results in a therapeutically significant lower level of pharmacological effectiveness and/or a therapeutically greater level of adverse physiological effects, e.g., greater liver toxicity.

The present invention is concerned generally with the field of pharmacology, specifically pharmacokinetics and toxicology, and more specifically with identifying and predicting differences in response to drugs in order to achieve superior efficacy and safety. It is further concerned with changes in RNA expressions due to specific events and interventions and with methods for determining and exploiting such differences to improve medical outcomes. Specifically, this invention describes the identification of changes in RNA expressions useful in the field of therapeutics for optimizing efficacy and safety of drug therapy by allowing prediction of pharmacokinetic and/or toxicologic behavior of specific drugs. Relevant pharmacokinetic processes include absorption, distribution, metabolism and excretion. Relevant toxicological processes include both dose related and idiosyncratic adverse reactions to drugs, including, for example, hepatotoxicity, blood dyscrasias and immunological reactions.

The levels of RNA expressions resulting from events or interventions that may be involved in the progression of disease and drug action are useful for determining drug efficacy and safety and for determining whether a given drug or other therapy may be safe and effective in an individual patient. Provided in the present invention are identifications of expressions which can be useful in connection with predicting differences in response to treatment and selection of appropriate treatment of a disease or condition. A target expression and variances have utility in pharmacogenetic association studies and diagnostic tests to improve the use of certain drugs or other therapies including, but not limited to, the drug classes and specific drugs identified in the 1999 Physicians' Desk Reference, 53^{rd} Edition, (Medical Economics Data, 1998), or the 1995 United States Pharmacopeia XXIII National Formulary XVIII (Interpharm Press, 1994), or other sources as described below.

Those familiar with drug use in medical practice will recognize that regulatory approval for drug use is commonly limited to approved indications, such as to those patients afflicted with a disease or condition for which the drug has been shown to be likely to produce a beneficial effect in a controlled clinical trial. Unfortunately, it has generally not been possible with current knowledge to predict which patients will have a beneficial response, with the exception of certain diseases such as bacterial infections where suitable laboratory methods have been developed. Likewise, it has generally not been possible to determine in advance whether a drug will be safe in a given patient. Regulatory approval for the use of most drugs is limited to the treatment of selected diseases and conditions. The descriptions of approved drug usage, including the suggested diagnostic studies or monitoring studies, and the allowable parameters of such studies, are commonly described in the “label” or “insert” which is distributed with the drug. Such labels or inserts are preferably required by government agencies as a condition for marketing the drug and are listed in common references such as the Physicians Desk Reference (PDR). These and other limitations or considerations on the use of a drug are also found in medical journals, publications such as pharmacology, pharmacy or medical textbooks including, without limitation, textbooks of nutrition, allopathic, homeopathic, and osteopathic medicine.

Many widely used drugs are effective in a minority of patients receiving the drug, particularly when one controls for the placebo effect. For example, the PDR shows that about 45% of patients receiving Cognex (tacrine hydrochloride) for Alzheimer's disease show no change or minimal worsening of their disease, as do about 68% of controls (including about 5% of controls who were much worse). About 58% of Alzheimer's patients receiving Cognex were minimally improved, compared to about 33% of controls, while about 2% of patients receiving Cognex were much improved compared to about 1% of controls. Thus a tiny fraction of patients had a significant benefit. Response to many cancer chemotherapy drugs is even worse. For example, 5-fluorouracil is standard therapy for advanced colorectal cancer, but only about 20-40% of patients have an objective response to the drug, and, of these, only 1-5% of patients have a complete response (complete tumor disappearance; the remaining patients have only partial tumor shrinkage). Conversely, up to 20-30% of patients receiving 5-FU suffer serious gastrointestinal or hematopoietic toxicity, depending on the regimen.

Thus, in a first aspect, the invention provides a method for analyzing changes in RNA expression for an individual patient suffering from a disease or condition to determine whether the changes are consistent with intended therapeutic effects given the current understanding of RNA function.

In a second aspect, the invention provides a method of analyzing changes in RNA expression in a group of individual patients suffering from a disease or condition to determine the likely clinical effects of an intervention in the general population.

In some cases, the intervention may incorporate selection of one or more from a plurality of medical therapies. Thus, the selection may be the selection of a method or methods which is/are more effective or less effective than certain other therapeutic regimens (with either having varying safety parameters). Likewise or in combination with the preceding selection, the selection may be the selection of a method or methods, which is safer than certain other methods of treatment in the patient.

The intervention may involve either positive selection or negative selection or both, meaning that the selection can involve a choice that a particular intervention would be an appropriate method to use and/or a choice that a particular intervention would be an inappropriate method to use. Thus, in certain embodiments, the presence of the at least one change in RNA expression indicative that the treatment will be effective or otherwise beneficial (or more likely to be beneficial) in the patient. Stating that the treatment will be effective means that the probability of beneficial therapeutic effect is greater than in a person not having the appropriate presence or absence of a particular change in RNA expression. In other embodiments, the presence of the at least one change in RNA expression is indicative that the treatment will be ineffective or contra-indicated for the patient. For example, a treatment may be contra-indicated if the treatment results, or is more likely to result, in undesirable side effects, or an excessive level of undesirable side effects. A determination of what constitutes excessive side-effects will vary, for example, depending on the disease or condition being treated, the availability of alternatives, the expected or experienced efficacy of the treatment, and the tolerance of the patient. As for an effective treatment, this means that it is more likely that desired effect will result from the treatment administration in a patient showing a change in RNA expression consistent with the desired clinical outcome. Also in preferred embodiments, the presence of the at least on change in RNA expression is indicative that the treatment is both effective and unlikely to result in undesirable effects or outcomes, or vice versa (is likely to have undesirable side effects but unlikely to produce desired therapeutic effects).

The invention may be useful in predicting a patient's tolerance to an intervention. In reference to response to a treatment, the term “tolerance” refers to the ability of a patient to accept a treatment, based, e.g., on deleterious effects and/or effects on lifestyle. Frequently, the term principally concerns the patients' perceived magnitude of deleterious effects such as nausea, weakness, dizziness, and diarrhea, among others. Such experienced effects can, for example, be due to general or cell-specific toxicity, activity on non-target cells, cross-reactivity on non-target cellular constituents (non-mechanism based), and/or side effects of activity on the target cellular substituents (mechanism based), or the cause of toxicity may not be understood. In any of these circumstances one may identify an association between the undesirable effects and variances in RNA expression.

Adverse responses to drugs constitute a major medical problem, as shown in two recent meta-analyses (Lazarou et al, “Incidence of Adverse Drug Reactions in Hospitalized Patients: A Meta-Analysis of Prospective Studies”, 279 JAMA 1200-1205 (1998); and Bonn, “Adverse Drug Reactions Remain a Major Cause of Death”, 351 LANCET 1183 (i998). An estimated 2.2 million hospitalized patients in the United Stated had serious adverse drug reactions in 1994, with an estimated 106,000 deaths (Lazarou et al.). To the extent that some of these adverse events are predictable based on changes in RNA expression, the identification of changes that are predictive of such effects will allow for more effective and safer drug use.

The present invention also has uses in the area of eliminating treatments. The phrase “eliminating a treatment” refers to removing a possible treatment from consideration, e.g., for use with a particular patient based on one or more changes in RNA expression, or to stopping the administration of a treatment which was in the course of administration.

Also in preferred embodiments, the method of selecting a treatment involves selecting a method of administration of a compound, combination of compounds, or pharmaceutical composition, for example, selecting a suitable dosage level and/or frequency of administration, and/or mode of administration of a compound. The method of administration can be selected to provide better, preferably maximum therapeutic benefit. In this context, “maximum” refers to an approximate local maximum based on the parameters being considered, not an absolute maximum. The term “suitable dosage level” refers to a dosage level which provides a therapeutically reasonable balance between pharmacological effectiveness and deleterious effects. Often this dosage level is related to the peak or average serum levels resulting from administration of a drug at the particular dosage level. Similarly, a “frequency of administration” refers to how often in a specified time period a treatment is administered, e.g., once, twice, or three times per day, every other day, once per week, etc. For a drug or drugs, the frequency of administration is generally selected to achieve a pharmacologically effective average or peak serum level without excessive deleterious effects (and preferably while still being able to have reasonable patient compliance for self-administered drugs). Thus, it is desirable to maintain the serum level of the drug within a therapeutic window of concentrations for the greatest percentage of time possible without such deleterious effects as would cause a prudent physician to reduce the frequency of administration for a particular dosage level.

RNA expression can be relevant to the treatment of more than one disease or condition, for example, RNA expression can have a predictive role in the initiation, development, course, treatment, treatment outcomes, or health-related quality of life outcomes of a number of different diseases, disorders, or conditions.

Standard recombinant DNA and molecular cloning techniques used here are well known in the art and are described by Sambrook et al., Molecular Cloning: A Laboratory Manual, Second Edition, (Cold Spring Harbor Laboratory Press, Cold Spring Harbor, N.Y., 1989) (hereinafter “Maniatis”); and by Silhavy et al., Experiments with Gene Fusions, (Cold Spring Harbor Laboratory Cold Press Spring Harbor, N.Y., 1984); and by Ausubel et al., Current Protocols in Molecular Biology, (Greene Publishing Assoc. and Wiley-Interscience, 1987).

The process of quantifying differences in RNA expressions involves extracting RNA from blood using a variety of techniques. See Jung et al, “Evaluation of Tyrosinase mRNA as a Tumor Marker in the Blood of Melanoma Patients”, 15(8) J CLIN ONCOL. 2826-2831 (August 1997).

The process of quantifying differences in RNA expressions involves comparing RNA extracted from two or more blood samples using genome-level analysis. Genome-level analysis facilitates looking at changes in gene expression of about 10,000 or more known genes to determine which expressions are most changed by an even or intervention. See Lockhart et al., “Expression Monitoring by Hybridization to High-Density Oligonucleotide Arrays. 14(13) NAT BIOTECHNOL., 1675-1680 (1996).

The analysis of large numbers of individuals to discover differences in RNA expression will result in better understanding of how changes in specific RNA expressions operate as a precursor to clinical changes in an individual. In identifying new patterns of RNA expression it is often useful to screen different population groups based on racial, ethnic, gender, and/or geographic origin because particular changes in RNA expression may differ in frequency between such groups.

It should be emphasized that it is currently not generally practical to study an entire population to establish the association between a specific disease or condition or response to a treatment and genetic expression. Such studies are preferably performed in controlled clinical trials using a limited number of patients that are considered to be representative of the population with the disease. Since drug development programs are generally targeted at the largest possible population, the study population will generally consist of men and women, as well as members of various racial and ethnic groups, depending on where the clinical trial is being performed. This is important to establish the efficacy of the treatment in all segments of the population.

The genes targeted as significant in the genome-level analysis are cross-referenced to databases that identify gene function. See Edgar et al, “Gene Expression Omnibus: NCBI Gene Expression and Hybridization Array Data Repository”, 30 NUCLEIC ACIDS RESEARCH 207-210 (2002).

Current genome-level analysis techniques many lack the specificity needed to complete the analysis. RNA extracted from two or more samples can also be compared by analyzing individual RNA transcripts using quantification real-time polymerase chain reaction and similar techniques to provide more detailed information on the patterns of change in gene expression in an individual or group of individuals over a time period. See Straub et al. “Quantitative Real-Time rt-PCR for Detection of Circulating Prostate-Specific Antigen mRNA Using Sequence-Specific Oligonucleotide Hybridization Probes in Prostate Cancer Patients”, 65 ONCOLOGY (Supplemental) 1:12-17 (2003).

In a preferred embodiment, results of the genome-level and individual RNA transcript analyses of samples before and after events or interventions to determine the effect of the event or the effectiveness of an intervention.

It is generally understood, administration of a particular treatment, e.g., administration of a therapeutic compound or combination of compounds, is chosen depending on the disease or condition which is to be treated. Thus, in certain preferred embodiments, the disease or condition is one for which administration of a treatment is expected to provide a therapeutic benefit.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

Effectiveness is measured in a particular population. In conventional drug development the population is generally every subject who meets the enrollment criteria (i.e. has the particular form of the disease or condition being treated). It is an aspect of the present invention that segmentation of a study population by genetic criteria can provide the basis for identifying a subpopulation in which a drug is effective against the disease or condition being treated.

The term “deleterious effects” refers to physical effects in a patient caused by administration of a treatment which are regarded as medically undesirable. Thus, for example, deleterious effects can include a wide spectrum of toxic effects injurious to health such as death of normally functioning cells when only death of diseased cells is desired, nausea, fever, inability to retain food, dehydration, damage to critical organs such as arrhythmias, renal tubular necrosis, fatty liver, or pulmonary fibrosis leading to coronary, renal, hepatic, or pulmonary insufficiency among many others. In this regard, the term “adverse reactions” refers to those manifestations of clinical symptomology of pathological disorder or dysfunction is induced by administration or a drug, agent, or candidate therapeutic intervention. In this regard, the term “contraindicated” means that a treatment results in deleterious effects such that a prudent medical doctor treating such a patient would regard the treatment as unsuitable for administration. Major factors in such a determination can include, for example, availability and relative advantages of alternative treatments, consequences of non-treatment, and permanency of deleterious effects of the treatment.

It is recognized that many treatment methods, e.g., administration of certain compounds or combinations of compounds, may produce side-effects or other deleterious effects in patients. Such effects can limit or even preclude use of the treatment method in particular patients, or may even result in irreversible injury, disorder, dysfunction, or death of the patient. Thus, in certain embodiments, the variance information is used to select both a first method of treatment and a second method of treatment. Usually the first treatment is a primary treatment which provides a physiological effect directed against the disease or condition or its symptoms. The second method is directed to reducing or eliminating one or more deleterious effects of the first treatment, e.g., to reduce a general toxicity or to reduce a side effect of the primary treatment. Thus, for example, the second method can be used to allow use of a greater dose or duration of the first treatment, or to allow use of the first treatment in patients for whom the first treatment would not be tolerated or would be contra-indicated in the absence of a second method to reduce deleterious effects or to potentiate the effectiveness of the first treatment.

In a related aspect, the invention provides a method for selecting a method of treatment for a patient suffering from a disease or condition by comparing change in gene to pharmacokinetic parameters, or organ and tissue damage, or inordinate immune response, which are indicative of the effectiveness or safety of at least one method of treatment.

Similar to the above aspect, in preferred embodiments, at least one method of treatment involves the administration of a compound effective in at least some patients with a disease or condition; the presence or absence of the at least one change in gene expression is indicative that the treatment will be effective in the patient; and/or the presence or absence of the at least one change in gene expression is indicative that the treatment will be ineffective or contra-indicated in the patient; and/or the treatment is a first treatment and the presence or absence of the at least one change in gene expression is indicative that a second treatment will be beneficial to reduce a deleterious effect or potentiate the effectiveness of the first treatment; and/or the at least one treatment is a plurality of methods of treatment. For a plurality of treatments, preferably the selecting involves determining whether any of the methods of treatment will be more effective than at least one other of the plurality of methods of treatment. Yet other embodiments are provided as described for the preceding aspect in connection with methods of treatment using administration of a compound; treatment of various diseases, and variances in genetic expressions.

In addition to the basic method of treatment, often the mode of administration of a given compound as a treatment for a disease or condition in a patient is significant in determining the course and/or outcome of the treatment for the patient. Thus, the invention also provides a method for selecting a method of administration of a compound to a patient suffering from a disease or condition, by determining changes in gene expression where such presence or absence is indicative of an appropriate method of administration of the compound. Preferably, the selection of a method of treatment (a treatment regimen) involves selecting a dosage level or frequency of administration or route of administration of the compound or combinations of those parameters. In preferred embodiments, two or more compounds are to be administered, and the selecting involves selecting a method of administration for one, two, or more than two of the compounds, jointly, concurrently, or separately. As understood by those skilled in the art, such plurality of compounds may be used in combination therapy, and thus may be formulated in a single drug, or may be separate drugs administered concurrently, serially, or separately. Other embodiments are as indicated above for selection of second treatment methods, methods of identifying changes in RNA expression, and methods of treatment as described for aspects above.

In another aspect, the invention provides a method for selecting a patient for administration of a method of treatment for a disease or condition, or of selecting a patient for a method of administration of a treatment, by analyzing changes in RNA expression as identified above in peripheral blood of a patient, where the changes in RNA expression is indicative that the treatment or method of administration that will be effective in the patient.

In one embodiment, the disease or the method of treatment is as described in aspects above, specifically including, for example, those described for selecting a method of treatment.

In another aspect, the invention provides a method for identifying patients with enhanced or diminished response or tolerance to a treatment method or a method of administration of a treatment where the treatment is for a disease or condition in the patient. The method involves correlating one or more changes in RNA expression as identified in aspects above in a plurality of patients with response to a treatment or a method of administration of a treatment. The correlation may be performed by determining the one or more changes in RNA expression in the plurality of patients and correlating the presence or absence of each of the changes (alone or in various combinations) with the patient's response to treatment. The changes in RNA expression may be previously known to exist or may also be determined in the present method or combinations of prior information and newly determined information may be used. The enhanced or diminished response should be statistically significant, preferably such that p=0.10 or less, more preferably 0.05 or less, and most preferably 0.02 or less. A positive correlation between the presence of one or more changes in RNA expression and an enhanced response to treatment is indicative that the treatment is particularly effective in the group of patients showing certain patters of RNA response. A positive correlation of the presence of the one or more expression changes with a diminished response to the treatment is indicative that the treatment will be less effective in the group of patients having those variances. Such information is useful, for example, for selecting or de-selecting patients for a particular treatment or method of administration of a treatment, or for demonstrating that a group of patients exists for which the treatment or method of treatment would be particularly beneficial or contra-indicated. Such demonstration can be beneficial, for example, for obtaining government regulatory approval for a new drug or a new use of a drug.

Preferred embodiments include drugs, treatments, variance identification or determination, determination of effectiveness, and/or diseases as described for aspects above or otherwise described herein.

In other embodiments, the correlation of patient responses to therapy according to changes in RNA expression is carried out in a clinical trial, e.g., as described herein according to any of the variations described. Detailed description of methods for associating variances with clinical outcomes using clinical trials is provided below. Further, in preferred embodiments the correlation of pharmacological effect (positive or negative) to changes in RNA expression in such a clinical trial is part of a regulatory submission to a government agency leading to approval of the drug. Most preferably the compound or compounds would not be approvable in the absence of this data.

As indicated above, in aspects of this invention involving selection of a patient for a treatment, selection of a method or mode of administration of a treatment, and selection of a patient for a treatment or a method of treatment, the selection may be positive selection or negative selection. Thus, the methods can include eliminating a treatment for a patient, eliminating a method or mode of administration of a treatment to a patient, or elimination of a patient for a treatment or method of treatment.

Also, in methods involving identification and/or comparison of changes in RNA expression, the methods can involve such identification or comparison for a plurality of genes. Preferably, the genes are functionally related to the same disease or condition, or to the aspect of disease pathophysiology that is being subjected to pharmacological manipulation by the treatment (e.g., a drug), or to the activation or inactivation or elimination of the drug, and more preferably the genes are involved in the same biochemical process or pathway.

As indicated above, many therapeutic compounds or combinations of compounds or pharmaceutical compositions show variable efficacy and/or safety in various patients in whom the compound or compounds is administered. Thus, it is beneficial to identify variances in RNA expressions. Thus, in a further aspect, the invention provides a method for determining whether a compound has a differential effect due to the presence or absence of at least one change in RNA.

The method involves identifying a first patient or set of patients suffering from a disease or condition whose response to a treatment differs from the response (to the same treatment) of a second patient or set of patients suffering from the same disease or condition, and then determining the differences in RNA expressions between the groups. A correlation between the presence or absence specific expression changes and the response of the patient or patients to the treatment indicates that the changes in RNAtic expression provide information about variable patient response. In general, the method will involve identifying at least one change in RNA expression.

The method can utilize a variety of different informative comparisons to identify correlations. For example a plurality of pairwise comparisons of treatment response and the presence or absence of at least one change in RNA expression can be performed for a plurality of patients.

Such methods can utilize either retrospective or prospective information concerning treatment response variability. Thus, in a preferred embodiment, it is previously known that patient response to the method of treatment is variable.

Also in preferred embodiments, the disease or condition is as for other aspects of this invention; for example, the treatment involves administration of a compound or pharmaceutical composition.

In preferred embodiments, the method involves a clinical trial, e.g., as described herein. Such a trial can be arranged, for example, in any of the ways described herein.

The present invention also provides methods of treatment of a disease or condition, preferably a disease or condition related to pharmacokinetic parameters, e.g. absorption, distribution, metabolism, or excretion, that affect a drug or candidate therapeutic intervention regarding efficacy and or safety, i.e. drug-induced disease, disorder or dysfunction or other toxicity effects or clinical symptomatology.

The present invention provides a method for treating a patient at risk for drug responsiveness, i.e., efficacy differences associated with pharmacokinetic parameters, and safety concerns, i.e. drug-induced disease, disorder, or dysfunction or diagnosed with organ failure or a disease associated with drug-induced organ failure. The methods include identifying such a patient and determining the patient's changes in genetic expressions. The patient identification can, for example, be based on clinical evaluation using conventional clinical metrics.

In a related aspect, the invention provides a method for identifying a patient for participation in a clinical trial of a therapy for the treatment of a disease, disorder, or dysfunction, or an associated drug-induced toxicity. The method involves determining the changes in genetic expression of a patient with (or at risk for) a disease, disorder, or dysfunction. The trial would then test the hypothesis that a statistically significant difference in response to a treatment can be demonstrated between two groups of patients each defined changes or lack of changes in genetic expression. Said response may be a desired or an undesired response. In a preferred embodiment, the treatment protocol involves a comparison of placebo vs. treatment response rates in two or more groups. For example a group with no changes in expression of one or more genes of interest may be compared to a group with changes in one or more gene expressions.

In another preferred embodiment, patients in a clinical trial can be grouped (at the end of the trial) according to treatment response, and statistical methods can be used to compare changes to gene expression in these groups. For example responders can be compared to nonresponders, or patients suffering adverse events can be compared to those not experiencing such effects. Alternatively response data can be treated as a continuous variable and the ability of gene expression to predict response can be measured. In a preferred embodiment, patients who exhibit extreme responses are compared with all other patients or with a group of patients who exhibit a divergent extreme response. For example if there is a continuous or semi-continuous measure of treatment response (for example the Alzheimer's Disease Assessment Scale, the Mini-Mental State Examination or the Hamilton Depression Rating Scale) then the 10% of patients with the most favorable responses could be compared to the 10% with the least favorable, or the patients one standard deviation above the mean score could be compared to the remainder, or to those one standard deviation below the mean score. One useful way to select the threshold for defining a response is to examine the distribution of responses in a placebo group. If the upper end of the range of placebo responses is used as a lower threshold for an ‘outlier response’ then the outlier response group should be almost free of placebo responders. This is a useful threshold because the inclusion of placebo responders in a ‘true’ response group decreases the ability of statistical methods to detect a changes in gene expression between responders and nonresponders.

In a related aspect, the invention provides a method for developing a disease management protocol that entails diagnosing a patient with a disease or a disease susceptibility, determining the changes in gene expression of the patient at a gene or genes correlated with treatment response and then selecting an optimal treatment based on the disease and the changes in gene expression. The disease management protocol may be useful in an education program for physicians, other caregivers or pharmacists; may constitute part of a drug label; or may be useful in a marketing campaign.

“Disease management protocol” or “treatment protocol” is a means for devising a therapeutic plan for a patient using laboratory, clinical and genetic data, including the patient's diagnosis and genotype. The protocol clarifies therapeutic options and provides information about probable prognoses with different treatments. The treatment protocol may provide an estimate of the likelihood that a patient will respond positively or negatively to a therapeutic intervention. The treatment protocol may also provide guidance regarding optimal drug dose and administration and likely timing of recovery or rehabilitation. A “disease management protocol” or “treatment protocol” may also be formulated for asymptomatic and healthy subjects in order to forecast future disease risks based on laboratory, clinical and gene expression variables. In this setting the protocol specifies optimal preventive or prophylactic interventions, including use of compounds, changes in diet or behavior, or other measures. The treatment protocol may include the use of a computer program.

In other embodiments of above aspects involving prediction of drug efficacy, the prediction of drug efficacy involves candidate therapeutic interventions that are known or have been identified to be affected by pharmacokinetic parameters, i.e. absorption, distribution, metabolism, or excretion. These parameters may be associated with hepatic or extra-hepatic biological mechanisms. Preferably the candidate therapeutic intervention will be effective in patients with the known changes in genetic expression but have a risk of drug ineffectiveness, i.e. nonresponsive to a drug or candidate therapeutic intervention.

In other embodiments, the above methods are used for or include identification of a safety or toxicity concern involving a drug-induced disease, disorder, or dysfunction and/or the likelihood of occurrence and/or severity of said disease, disorder, or dysfunction.

In other embodiments, the invention is suitable for identifying a patient with non-drug-induced disease, disorder, or dysfunction but with dysfunction related to aberrant enzymatic metabolism or excretion of endogenous biologically relevant molecules or compounds.

### EXAMPLES

### Example 1

Clinical Trial of an Experimental Drug The study involves the collection of Total RNA in a clinical setting, genome-level identification of gene expression changes, high-resolution temporal analysis of specific genes of interest, and correlation of results to clinical data.

Genome-Level Analysis: RNA will be extracted and preserved from the peripheral blood (PBMCS) of five participants, four of whom will be given a common over-the-counter medication and one of whom being a control. RNA from each patient will be collected at T−1 hour, T, and T+1 hour, for a total of 15 RNA samples, with T being the time of dosing.

The RNA samples will be subjected to genome-level analysis to determine 1) which of the five patients is the control patient, 2) which 10 genes are most effected by the drug and 3) what type of medication has been given to the patients given current knowledge of genetic pathway functions.

Gene-Specific Analysis: RNA will be extracted and preserved from the peripheral blood (PBMCs) of 25 participants, 20 of whom will be given the same common over-the-counter medication (the drug) and 5 of whom being controls. RNA from each patient will be collected at T, T+30 minutes, T+1 hour, T+2 hours, and T+4 hours for a total of 125 RNA samples, with T being the time of dosing.

The 125 RNA samples will be subjected to gene-specific analysis for the 10 genes identified during the genome-level analysis. The analysis will determine 1) which five subjects are the controls, 2) the average time of initial effect of the drug, and 3) the time of the maximum effect of the drug.

The study will primarily allow us to gain experience using genomic techniques for clinical trials. Secondarily, the results from the genomic analysis will be compared to clinical data to determine the applicability of current genomic techniques for clinical trials. This protocol describes a clinical study involving human subjects for the purpose of using comparative mRNA expression quantification as a precursor to clinical symptoms in clinical trials.

Not every gene is turned on (or expressed). For example, we do not want our brain cells to make hemoglobin, the protein required to carry oxygen around in our blood. The genes in the brain that will ultimately make red blood cells would not be expressed. mRNA is created only when genes are expressing. mRNA levels in cells routinely change as different genes express and then stop expressing. Different genes express at different times of the day (controlling our biological clock), at different times of the month (controlling menstruation in woman), and as we age (controlling virtually every aspect of the aging process). mRNA levels also change due to disease and external events. mRNA to create tumors will only be present when a gene is expressing for cancer. mRNA to initiate swelling in joints will be present at higher levels when a person has rheumatoid arthritis then when that person does not. Recent advances in medical technology allow us to measure the amount of mRNA in cells. This protocol incorporates two currently available technologies, GeneChips (Affymetrix) and ArrayPlate (High Throughput Genomics) to measure the levels of mRNA in biological samples. However, the quantification technologies may change over time, and the present invention is not limited to any particular technology.

Background of the Protocol The protocol compares mRNA levels in the same subjects at different points in time. The use of a comparative technique avoids two problems: 1) The process of normalizing samples is exceedingly difficult. 2) It may be difficult (perhaps ultimately impossible) to determine what specific level of mRNA is needed to trigger a clinical response. We avoid needing to normalize samples using the comparative technique as we are already able to draw the conclusion that larger changes in mRNA levels are more likely to trigger clinical responses than smaller changes.

The protocol uses a two-step quantification process, also to help eliminate some of the current problems with gene expression. There are between 30,000 and 40,000 coding genes. Running detailed gene-specific analysis on these genes, both separately and in combination, would be very costly and generate more data than can routinely be: analyzed.

The use of only a genome-level analysis (where thousands of genes are analyzed at the same time) is also problematic. Genomic-level analyses do not have the accuracy or the reliability of gene specific processes. Therefore, a genome-level analysis is undertaken to identify those genes most changed between samples. A more detailed analysis using gene-specific analyses is then done on those genes of interest.

Objectives
1. Total RNA can be collected in a clinical setting, prepared and sent for genomic analysis in a manner similar to how other clinical samples are now sent to reference laboratories for clinical analysis.
2. Whole genome analysis of changes in gene expressions can be used to identify patients given a common over-the-counter medication as compared to those who have not.
3. Whole genome analysis of blood samples can identify the expression of 10 genes most changed by an over-the-counter medication.
4. Analysis of the 10 genes determined by the whole genome analysis as most changed by an over the counter medication will identify what type of over-the-counter medication was given to the subjects.
5. Gene-specific analysis of 10 genes of interest can identify patients given an over-the-counter medication as compared to those who have not.
6. Gene-specific analysis of 10 genes of interest can identify the time it takes for a common over the counter medication to begin to metabolize.
7. Gene-specific analysis of 10 genes of interest can identify the time of maximum effect of a common over the counter medication.
8. The results of the gene-specific analysis are consistent with the known clinical effects of the medication.
   Study Design and Methods: Whole Genome Analysis

Inclusion Criteria
Normal, healthy donors. Caucasian males age 50-60.
Patients must fast for 8 hours prior to commencing the study. Patients may drink bottled (not tap) water only during the 8 hour period.

[Note: Females are excluded due to the need to coordinate menstrual cycles in females. The effect of race on these studies has not yet been determined. A 10 year age group is used to minimize genetic effects due to aging.]

Exclusion Conditions
Patients known or believed to be reactive for HIV 1/2, HCV, HBsAg.
Donors that in the last 48 hours have consumed alcohol.
Donors that in the last 48 hours have taken depressants (hypnotics, sedatives, tranquilizers, etc.) or MAO inhibitors.
A history of lower respiratory disease including asthma, increased intraocular pressure, hyperthyroidism, cardiovascular disease or hypertension.
Known hypersensitivity to diphenhydramine hydrochloride (Benadryl) and other antihistamines of similar chemical structure.
Donors with narrow-angle glaucoma, stenosing peptic ulcer, pyloroduodenal obstruction, symptomatic prostatic hypertrophy, or bladder-neck obstruction.
Smokers, users of tobacco of any kind.

Study Protocol—Whole Genome Analysis
The participants in the study present themselves at 10:00 am.
Participants can drink filtered (not tap) water during the study.
Participants cannot be exposed to sunlight during the duration study.
Draw 8 ml blood per patient per the Whole Genome Draw Protocol.
Wait 60 minutes.
Draw 8 ml blood per patient per the Whole Genome Draw Protocol.
Administer 50 mg drug to drug group within 2 minutes of 2nd blood draw.
Draw 8 ml blood per patient per the Whole Genome Draw Protocol 1 hour after administration of drug.
Offer participants light snack, verify that they are not impaired, and release the participants.

Whole Genome Draw Protocol
Use BD Vacutainer Cell Preparation Tube at room temperature (18-25° C.). Anticoagulant is Heparin.
Label tube with Protocol Number, patient ID, draw date and time.
Collect whole blood using generally accepted procedures and precautions including the prevention of backflow. Collect 8 ml per tube.
Gently invert the tubes 8 times to assure uniform mixture of the whole blood.
After collection, immediately centrifuge samples at room temperature using the Fisher Scientific Model 3200 R centrifuge at 1800 RCF for 30 minutes. Allow the centrifuge to stop without braking or manual intervention.

Note: After centrifugation, the plasma will rise to the top of the tube. The mononuclear cells will be in a whitish layer just under the plasma layer. Each CPT Tube will yield approximately 1×10⁷ mononuclear cells and 10-20 μg Total RNA.
Invert the unopened CPT Tube gently 5 to 0.10 times until the mononuclear cells begin to suspend in the plasma. The mixture does not need to be uniform.
Send to Expression Analysis for RNA extraction and Whole Genome analysis in insulated container containing water ice by FedEx Priority delivery.

Whole Genome Analysis (Expression Analysis)
Extract Total RNA from the 15 samples per standard protocol.
Employ standard inventory control measures to manage array receipt, use and expiration dates.
Starting with 10 micrograms to total RNA, perform a quality assurance test of the Total RNA using the Agilent BioAnalyzer.
Develop the cRNA probe.
Hybridize, stain, wash and scan your cRNA probe onto Genechip® array
Provide expression results in electronic format to BRS. The standard Affymetrix output files delivered for each sample will be: *.EXP (experimental information file)*.DAT file (image of scanned probe array), *.CEL file (averaged cell intensities), *.CHP (analysis output file), *.RPT (analysis report file). These files are readable using the Affymetrix Microarray Analysis Suite software. All resultant data will be returned via express delivery on a CD ROM and electronically via EA server secure link for a period of thirty (30) days following the completion of all EA testing and analysis.
Return all quality control test results to BRS.
Convert the raw Affymetrix output files (five per sample) into useable formats: tabulated expression intensity estimates in text files and/or Excel formats, summarized for all samples in client experiment.

Tabulate and summarize results by treatment groups in study designs that have the samples grouped into two or more subsets (e.g., treatment versus control, or multiple treatments). Summaries by treatment will include text files recording standard summary statistics, including mean and SE of gene intensities within treatments groups, and mean and SE of ranks of each gene within treatment groups.

Data Format - Whole Genome Analysis

| | | SE Delta | Mean Fold | SE Fold | Mean Fold | Mean Fold |
|---|---|---|---|---|---|---|
| | Delta Fold | Fold | Change | Change | Change | Change |
| Gene Code | Change | Change | T to T + 1 hr | T to T + 1 hr | T − 1 hr to T | T − 1 hr to T |
| 1 | +/−X.XX | +/−X.XX | +/−X.XX | +/−X.XX | +/−X.XX | +/−X.XX |

Study Protocol—Gene-Specific
The participants in the study present themselves at 9:00 am.
Participants can drink filtered (not tap) water during the study.
Participants cannot be exposed to sunlight during the duration study.
Draw 8 ml blood per patient per the Gene-Specific Draw Protocol.
Administer drug to drug group within 2 minutes of blood draw.
Draw 8 ml blood per patient per the Whole Genome Draw Protocol 30 minutes, 1 hour, 2 hours, and 4 hours, after administration of drug.
Offer participants light snack, verify that they are not impaired, and release the participants.

Whole Genome Draw Protocol
Use BD Vacutainer Tube at room temperature (18-25 C). Anticoagulant is Heparin.
Label tube with Protocol Number, patient ID, draw date and time.
Collect whole blood using generally accepted procedures and precautions including the prevention of backflow. Collect 8 ml per tube.
Gently invert the tubes 8 times to assure uniform mixture of the whole blood.
Process according to HTG-supplied protocol.

Gene-Specific Analysis (High Throughput Genomics)
HTG will measure 10 target genes selected by BRS plus 4 invariant housekeeping genes, selected by mutual agreement, in same well, on the same sample in replicates of 8 (eight).
Report to include: Statistical analysis to identify and eliminate any outliers among the assay replicates, and among the patients within each treatment group; Signal intensity with standard deviation and standard error of the mean; Signal intensity normalized to the housekeeping genes with standard deviation and standard error of the mean; Provide graphical time course data (each graph with 10 lines, one line per target gene, showing standard deviation for each observation); Provide Excel file of raw data and analyzed data.

Bioinformatics
Give each patient a donor number.
Maintain a record of the donor number and the patient that number represents internally within your office.
Complete the Case Report Form. Include the form with the donation.
Label Tubes and case report forms as follows: TranscriptPanel 001; Patient ID; Collection Date; Collection Time

Analysis of the Study

The Whole Genome analysis will primarily incorporate standard statistical measures (mean and standard error) to determine the most changed genes. The HumanCyc database will then be consulted to determine the function of the 10 most changed genes. The Whole Genome analysis will be considered successful if the 10 most changed genes are known to have an association with histamine (such as H1, H2 and H3), Parkinson's disease (tremors, etc.), motion sickness, or the sleep functions. However, it is likely that the study will uncover other genetic effects of the drug.

The gene-specific analysis will be considered successful of the maximum genetic effect occurs at or before T+1 hour and there is a residual effect at residual effect at T+4 hours.

### Example 2

### Gene Expression Profiles

Transcript Panels as described herein are concerned with the field of pharmacology, specifically pharmacogenomics, and more specifically with identifying and predicting differences in genomic response to drugs in order to achieve superior efficacy and safety. It is further concerned with changes in RNA expressions due to specific events and interventions and with methods for determining and exploiting such differences to improve medical outcomes.

Transcript Panels describe the identification of changes in RNA expressions useful in the field of therapeutics for optimizing efficacy and safety of drug therapy by allowing prediction of pharmacokinetic and/or toxicologic behavior of specific drugs. Relevant pharmacokinetic processes include absorption, distribution, metabolism and excretion. Relevant toxicological processes include both dose related and idiosyncratic adverse reactions to drugs, including, for example, hepatotoxicity, blood dyscrasias and immunological reactions.

Changes in RNA expressions resulting from events or interventions that may be involved in the progression of disease and drug action are useful for determining drug efficacy and safety and for determining whether a given drug or other therapy may be safe and effective in an individual patient. Provided in this invention are identifications of expressions which can be useful in connection with predicting differences in response to treatment and selection of appropriate treatment of a disease or condition. A target expression and variances have utility in pharmacogenetic association studies and diagnostic tests to improve the use of certain drugs or other therapies including, but not limited to, the drug classes and specific drugs identified in the 1999 Physicians' Desk Reference, 53rd edition, (Medical Economics Data, 1998) or the 1995 United States Pharmacopoeia XXIII National Formulary XVIII, (Interpharm Press, 1994), or other similar sources.

Transcript Panels provide a method for analyzing changes in RNA expression for an individual patient suffering from a disease or condition to determine whether the changes are consistent with intended therapeutic effects given the current understanding of gene function. Transcript Panels also provide a method of analyzing changes in RNA expression in a group of individual patients suffering from a disease or condition to determine the likely clinical effects of an intervention in the general population.

TABLE 1
Genes Evaluated in the Study

| Symbol | LocusID | Name |
|---|---|---|
| IL10 | 3586 | interleukin 10 |
| EDN1 | 1906 | endothelin 1 |
| GATA2 | 2624 | GATA binding protein 2 |
| IL15 | 3600 | interleukin 15 |
| IL2 | 3358 | interleukin 15 |
| CD86 | 942 | CD86 antigen |
| ICAM1 | 3383 | intercellular adhesion molecule 1 (CD54) |
| CD83 | 9308 | CD83 antigen |
| MHC2TA | 4261 | MHC class II transactivator |
| IFNA1 | 3439 | interferon, alpha 1 |

TABLE 2
Relative Expression Coefficient

| Symbol | Relative Expression Coefficient |
|---|---|
| IL10 | 1.00 |
| EDN1 | 0.87 |
| GATA2 | 0.85 |
| IL15 | 0.77 |
| IL2 | 0.65 |
| CD86 | 0.65 |
| ICAM1 | 0.62 |
| CD83 | 0.57 |
| MHC2TA | 0.55 |
| IFNA1 | 0.53 |

Gene Identification 10 genes most changed by the event or intervention were identified. (See Table 1 for the Symbol, LocusID and name of the 10 genes.)

The relative expression changes for the 10 genes of interest were determined. (See Table 2 for the relative expression coefficient.) The relative expression coefficient identifies the importance of the gene in the subsequent analyses.

The functions of the genes were identified based on currently available information. (See Table 3 for a description of the gene function.)

Expression Levels The pattern of gene expression for the 10 genes of interest was recorded prior to and for 4 hours after the event or intervention. [FIG. 1] shows the relative expression levels where T is the time of the event or intervention.

TABLE 3
Gene Function

| Symbol | Gene Function |
|---|---|
| IL10 | Pinderski Oslund et al. (1999) found that IL10 blocks atherosclerotic events in |
| | vitro and in vivo. Terkeltaub (1999) suggested that IL10 may arrest and |
| | reverse the chronic inflammatory response in established atherosclerosis. |
| EDN1 | Endothelin-1 is a pain mediator that is involved in the pathogenesis of pain |
| | states ranging from trauma to cancer. It is a potent vasoactive peptide and |
| | appears to be implicated in the pathogenesis of pain associated with ischemic |
| | states (such as coronary artery disease or sickle cell anemia), and |
| | inflammation (such as arthritis) in addition to cancer. (Inoue et al., 1989). |
| GATA2 | Tong et al. (2000) concluded that GATA2 and GATA3 regulate adipocyte |
| | differentiation through molecular control of the preadipocyte-adipocyte |
| | transition. |
| IL15 | Roberts et al. (2001) concluded that NKG2D can function as a potent |
| | costimulator of TCR-mediated activation of IELs. In addition, they suggested |
| | that IL15, which is secreted by intestinal epithelial cells upon inflammation or |
| | viral infection, can induce excessive NKG2D expression if uncontrolled, |
| | leading to the development of autoimmune disease against MICA (600169)-/ |
| | MICB (602436)-expressing epithelial cells. |
| IL2 | The protein encoded by this gene is a secreted cytokine that is important for |
| | the proliferation of T and B lymphocytes. Since interleukin-2 and interleukin- |
| | 2 receptor act as required for the proliferation of T cells, defects in either the |
| | ligand or the receptor would be expected to cause severe combined |
| | immunodeficiency. The targeted disruption of a similar gene in mice leads to |
| | ulcerative colitis-like disease, which suggests an essential role of this gene in |
| | the immune response to antigenic stimuli. |
| CD86 | Eosinophils activated by IL3 may contribute to T-cell activation in allergic |
| | and parasitic diseases by presenting superantigens and peptides to T |
| | cells. 11714768 |
| ICAM1 | Expression of HLA-DR antigen (see 142860) and ICAM1 in human |
| | conjunctival epithelium is upregulated in patients with dry eyes associated |
| | with Sjogren syndrome (270150) |
| CD83 | Using flow cytometric analysis, Scholler et al. (2001) showed that CD83 |
| | binds to monocytes but not lymphocytes and that the binding is enhanced by |
| | stress. Immunoprecipitation and immunoblot analysis indicated that CD83 |
| | binds to a 72-kD ligand containing sialic acid. Scholler et al. (2001) concluded |
| | that CD83 is an adhesion receptor belonging to the SIGLEC family (see |
| | 600751). |
| MHC2TA | MHC2TA encodes a non-DNA binding transactivator that functions both in |
| | constitutive and inducible MHC Class II expression. Mutations in MHC2TA |
| | result in the complementation group A of Bare Lymphocyte Syndrome. |
| IFNA1 | Deficiency of production of immune interferon, associated with absent natural |
| | killer (NK) activity, was described in a child with persistent Epstein-Barr |
| | virus infection who developed a fatal lymphoproliferative disorder described |
| | other children with deficient production of immune interferon; all had |
| | markedly depressed NK activity. |

Initial Effect Aggregate genetic activity begins in the period T+30 minutes and T+1 hour. Activity regarding IL10, CD86, ICAM1, MHC2TA and EDN1 begins at T+30 minutes with expression of the remaining genes altered by T+1 hour.

Maximum Genetic Activity Aggregate maximum genetic activity occurs during the period T+1 hour and continues through T+3 hours.

Residual Genetic Activity Most genetic activity is returning to pre-event levels by T+4 hours.

Pharmacological Effects The event or intervention shows potential usefulness in the areas of anti-inflammatory and immunosuppressive responses. Of particular interest may be effectiveness in asthma and other bronchioconstrictive diseases.

The event or intervention also affects immune responses and the production of T and B cells. Interestingly, most of the immune-related genes associated with Multiple Sclerosis are affected by this event or intervention. (Filion et al., “Monocyte-Derived IL12, CD86 (B7-2) and CD40L Expression in Relapsing and Progressive Multiple Sclerosis”, 106(2) CLIN IMMUNOL., 127-138 (February, 2003)).

IL-10 IL-10 showed significant up regulation from T+30 minutes through T+4 hours with residual effect continuing at T+4 hours. Review of available research indicates mostly positive indications due to the up regulation of IL-10. Up regulation of IL-10 is associated with anti-inflammatory and immunosuppressive effects. (Bartz et al., “Respiratory Syncytial Virus Induces Prostaglandin E2, IL-10 and IL-11 Generation in Antigen Presenting Cells” 129(3) CLIN EXP IMMUNOL. 438-445 (September, 2002)). Specifically, up regulation of IL-10 is believed to: (1) Reduce susceptibility to Epstein-Barr Virus and associated nasopharyngeal cancer (NPC) (Du et al., “Endogenous Expression of Interleukin-8 and Interleukin-10 in Nasopharyngeal Carcinoma Cells and the Effect of Photodynamic Therapy”, 10(1) INT J MOL MED., 73-76 (July 2002)); (2) Reduce incidents of irreversible septic shock after cecal ligation and puncture (CLP); (3) Reduced expression of granulocyte-macrophage colony-stimulating factor (GM-CSF) in non-small cell lung cancer (NSCLC) (Egi et al., “Upregulation of Intragraft Interleukin-10 by Infusion of Granulocyte Colony-Stimulating Factor-Mobilized Donor Leukocytes”, 15(9-10) TRANSPL INT., 479-485 (October, 2002); (E-pub. 24 Sep. 2002)); (4) Decreased persistence of B. quintana (Capo et al., “Bartonella Quintana Bacteremia and Overproduction of Interleukin-10: Model of Bacterial Persistence in Homeless People”, 187(5) J INFECT DIS., 837-844 (March, 2003); (E-pub 24 Feb. 2003)). Risks associated with up-regulation of IL10 include Decreased tolerance to implants (graft vs. host disease).

EDN1 EDN1 showed significant down regulation from T+30 minutes and mostly ending by T+4 hours. Review of available research indicates positive indications due to the down regulation of EDN1. Down regulation of EDN1 is associated with reduced vasoconstriction and reduced bronchioconstriction. Specifically, down regulation of EDN1 may be associated with (1) Reduced bone metastases in prostate and breast cancer patients. (Yin et al., “A Causal Role for Endothelin-1 in the Pathogenesis of Osteoblastic Bone Metastases”, 100(19) PROC NATL ACAD SCI USA. 10954-10959 (September, 2003) (E-pub 26 Aug. 2003); (Comment in 100(19) PROC NATL ACAD SCI USA., 10588-10589 (September, 2003)); (2) Reduced bronchioconstriction in asthmatic children. (E1-Gamal et al., “Plasma Endothelin-1 Immunoreactivity in Asthmatic Children”, 88(4) ANN ALLERGY ASTHMA IMMUNOL. 370-373 (April, 2002); (Comment in 88(4) ANN ALLERGY ASTHMA IMMUNOL., 345-346 (April 2002)); (3) Reduced antiapoptotic activity of endothelin for melanoma cells. (Eberle et al., “Endothelin-1 Decreases Basic Apoptotic Rates in Human Melanoma Cell Lines”, 119(3) INVEST DERMATOL., 549-555 (September 2002)); (4) Reduced tumor growth rates. (Li et al., “Endothelin-1 Expression and Quantitative Analysis in Astrocytomas” [Article in Chinese], 21(10) AI ZHENG, 1109-1111 (October, 2002)); (5) Reduction in progression of osteoarthritis. (Roy-Beaudry et al., “Endothelin 1 Promotes Osteoarthritic Cartilage Degradation via Matrix Metalloprotease 1 and Matrix Metalloprotease 13 Induction”, 48(10) ARTHRITIS RHEUM., 2855-2864 (October, 2003)). There are no known risks associated with the down regulation of EDN1.

GATA-2 GATA-2 showed moderate up regulation from T+30 minutes through T+3 hours. Review of available research indicates that GATA-2 is crucial for the maintenance and proliferation of immature hematopoietic progenitors. (Ohneda K. and M. Yamamoto, “Roles of Hematopoietic Transcription Factors GATA-1 and GATA-2 in the Development of Red Blood Cell Lineage”, 108(4) ACTA HAEMATOL. 237-245 (2002)). There are no reports of risks associated with up regulation of GATA2.

IL-15 IL-15 showed moderate down regulation from T+30 minutes to T+2 hours. Review of available research indicates that IL-15 has many biological functions. It can play a role in the initiation and outcome of acute and chronic organ transplant rejection. There is a potential for down regulation of IL-15 to hinder the T-cell response to human intracellular pathogens. Further, reduced IL-15 expression may contribute to the pathogenesis of atopic dermatitis.

IL-2 IL-2 showed moderate up regulation from T+30 minutes with continuing effect past T+4 hours. IL-2 plays an important and complex role in the immune system, serving as a growth factor, a differentiation factor, and a regulator of cell death. (B. H. Nelson, “Interleukin-2 Signaling and the Maintenance of Self-Tolerance”, 5 CURR DIR AUTOIMMUN., 92-112 (2002)). IL-2 plays similar roles to IL-15 (stimulating the production of T cells for example) so it is somewhat of a curiosity that IL-15 is down regulated while IL-2 is up regulated.

Up regulation of IL-2 potentially improves the effect of Taxol and other cytotoxic agents. (Bonhomme-Faivre et al., “Recombinant Interleukin-2 Treatment Decreases P-glycoprotein Activity and Paclitaxel Metabolism in Mice”, 13(1) ANTICANCER DRUGS, 51-57 (January, 2002)). T cells deprived of IL-2 undergo apoptosis generally. (Devireddy L. R. and M. R. Green, “Transcriptional Program of Apoptosis Induction Following Interleukin 2 Deprivation: Identification of RC3, a Calcium/Calmodulin Binding Protein, as a Novel Proapoptotic Factor”, 23(13) MOL CELL BIOL., 4532-4541 (July, 2003)).

CD86 Up regulation of CD86 has been shown to increase the activity of B cells. (Suvas et al., “Distinct Role of CD80 and CD86 in the Regulation of the Activation of B Cell and B Cell Lymphoma”, 277(10) J BIOL CHEM., 7766-7775 (March, 2002); (E-pub 28 Nov. 2001)).

ICAM-1 ICAM-1 is initially moderately down regulated and then moderately up regulated. This suggests that the event or intervention may initially reduce inflammation but then cause some inflammation. ICAM-1 is involved in the regulation of allergic inflammation and may reflect the severity of inflammation in the airway of asthmatic patients. (Kokuludag et al., “Elevation of Serum Eosinophil Cationic Protein, Soluble Tumor Necrosis Factor Receptors and Soluble Intercellular Adhesion Molecule-1 Levels in Acute Bronchial Asthma”, 12(3) J INVESTIG ALLERGOL CLIN IMMUNOL., 211-214 (2002)). Up regulation of ICAM-1 may play an important role in the pathogenic process of corpulmonale. (Chen et al., “Study of the Function of Leukocyte Adhesion Molecules in Chronic Respiratory Diseases” [Article in Chinese], 25(2) ZHONGHUA JIE HE HE HU XI ZA ZHi., 94-97 (February, 2002)).

CD83 CD83 is somewhat up regulated as a result of the event or intervention. CD83 is a marker gene for mature dendric cells (DC). The infiltration of tumors by mature DC expressing CD83 may be of great importance in initiating the primary anti-tumor immune response. Other studies implicate CD83 in immune response.

MCH2TA This gene was found to have diverse functions, which could impact Ag processing, signaling, and proliferation. (Nagarajan et al., “Modulation of Gene Expression by the MHC Class II Transactivator”, 169(9) J IMMUNOL., 5078-5088 (November, 2002)). Significant down regulation results in serious immuno-deficiencies. (Dziembowska et al., “Three Novel Mutations of the CIITA Gene in MHC Class II-Deficient Patients with a Severe Immunodeficiency”, 53(10-11) IMMUNOGENETICS, 821-829 (February, 2002); (E-pub 29 Jan. 2002)).

IFNA1 Results on this gene are inconclusive.

### Example 3

### Genetic Expression Monitoring and Intervention

The APC I1307K mutation is associated with colorectal cancer. Specifically, germ-line deletions at APC codons 1061, 1068, and 1309 increase the risk of FAP.

RNA will be extracted and preserved from the peripheral blood (PBMCs) of individuals known to have APC germ-line deletions as a result of commercially available genetic tests for APC mutations. RNA will be collected and extracted monthly.

RNA will be quantified using allele-specific real-time reverse transcription PCR or similar techniques to determine relative expression levels of mRNA coding from the mutated APC genes.

The protocol will allow us to gain experience using genomic techniques for assessing the progression of colon cancer prior to the onset of clinical symptoms. This protocol describes a clinical study involving human subjects for the purpose of using comparative mRNA expression quantification as a precursor to clinical symptoms the progression of disease.

The same Background of the Protocol of Example 1, supra, would be applicable.

Objectives
1. Total RNA can be collected in a clinical setting, prepared and sent for genomic analysis in a manner similar to how other clinical samples are now sent to reference laboratories for clinical analysis.
2. Analysis of the quantification levels of mutated APC genes offers a prediction on the likelihood and timing of the onset of clinical symptoms.
   Study Design and Methods

Inclusion Criteria
Patients known positive for APC mutations implicated in FAP by commercial testing.

Exclusion Conditions
Patients known or believed to be reactive for HIV 1/2, HCV, HBsAg.
Smokers, users of tobacco of any kind.

Study Protocol
Patients cannot have consumed alcohol, caffeine, or over the counter medication in the 48 hours prior to having blood drawn.
Patients must have fasted for 8 hours prior to having blood drawn.
The participants in the study present themselves at 10:00 am.
Draw 8 ml blood per patient per the Draw Protocol.

Draw Protocol
Use 8 ml BD Cell Preparation tube at room temperature (18-25° C.). Anticoagulant is Sodium Citrate.
Label tube with Protocol Number, patient ID, draw date and time.
Collect whole blood using generally accepted procedures and precautions including the prevention of backflow. Collect 8 ml per tube.
Gently invert the tubes 8 times to assure uniform mixture of the whole blood.
After collection, immediately centrifuge samples at room temperature using the Fisher Scientific Model 3200 R centrifuge at 1800 RCF (1,650×g) for 30 minutes. Allow the centrifuge to stop without braking or manual intervention.

Note: After centrifugation, the plasma will rise to the top of the tube. The mononuclear cells will be in a whitish layer just under the plasma layer. Each CPT Tube will yield approximately 1×10⁷ mononuclear cells and 10-20 μg Total RNA.
Discard the plasma layer leaving a security layer of plasma above the mononuclear cells.
Transfer the mononuclear cells to a 15 ml falcon tube. Fill with 1×PBS.
Centrifuge at 500×g for 10 minutes.
Discard supernatant.
Add 1 ml TRIzol Reagents (Invitrogen, CA). Lyse cells by repeated pipetting and incubate at room temperature for 5 minutes.
Store at −80 C.

Gene-Specific Analysis Genotypes were determined at nine different polymorphic marker loci. The following markers were intragenic to APC: (i) an A/G polymorphism (National Center for Biotechnology Information single-nucleotide polymorphism cluster ID: rs2019720) located within the promoter region, (ii) an A/T polymorphism (rs1914) located within intron 7, (iii) a T/C polymorphism located within exon 11, (iv) an A/G polymorphism located within exon 15I (Sieber et al., “Whole-Gene APC Deletions Cause Classical Familial Adenomatous Polyposis, But Not Attenuated Polyposis or ‘Multiple’ Colorectal Adenomas”, 99(5) PROC. NATL. ACAD. SCI. USA. 2954-2958 (2002)), (v) an A/G polymorphism located within exon 15J (Sieber et al., 2002, supra), and (vi) a T/C polymorphism located within the 3′ untranslated region (Sieber et al., 2002, supra). In addition, all patients with identified APC deletions were genotyped for two polymorphic markers, an A/G (rs748628) and a C/T polymorphism (rs1922665) located about 110 kb and 37 kb 5′ of APC, as well as three microsatellite markers, D5S346, D5S656, and D5S421 located about 32 kb, 396 kb, and 628 kb 3′ of APC, respectively (University of California, Santa Cruz Genome Browser, Apr. 1, 2001 freeze at http://genome.ucsc.edu:

PROBE SEQUENCES

| | |
|---|---|
| APC exon 14 forward | GCCAGACAAACACTTTAGCCATTA |
| | (SEQ ID NO. 1) |
| APC exon 14 reverse | TACCTGTGGTCCTCATTTGTAGCTAT |
| | (SEQ ID NO. 2) |
| APC exon 14 probe | CTGGACACATTCCGTAATATCCCACCTCC |
| (5′-FAM, 3′-TAMRA) | (SEQ ID NO. 3) |
| PRIMER SEQUENCES | |
| rs748628 | CTTTTCTTTTTCTTTTTC[t]T |
| | CTTTTCTTTTTCTTTTTC[c]C |
| | CTTACTACATTCAAGGGGAT |
| | (SEQ ID NO. 4) |
| rs1922665 | CTTCCCTGTTCTGCCAATCT |
| | GCACTGGATGTTCAGAGACG |
| | TCTGTTGGTGGTCTCC |
| | (SEQ ID NO. 5) |
| Promoter | TGGGGATGAGAGAAAGAGGAGGA |
| | CGCAAAAAGCCACTACCACTG |
| | (SEQ ID NO. 6) |
| Intron7 | CAGGTTTGAGCCATCATGC |
| | ATCCAATCCCTAAGCTTGACTG |
| | (SEQ ID NO. 7) |
| Exon 11 | GATGATTGTCTTTTTCCTCTTGC |
| | CTGAGCTATCTTAAGAAATACATG |
| | (SEQ ID NO. 8) |
| Exon 15I | AGTAAATGCTGCAGTTCAGAGG |
| | CCGTGGCATATCATCCCCC |
| | (SEQ ID NO. 9) |
| Exon 15J | CCCAGACTGCTTCAAAATTACC |
| | GAGCCTCATCTGTACTTCTGC |
| | (SEQ ID NO. 10) |
| 3′Untranslated region | GCATTAAGAGTAAAATTCCTCTTAC |
| | ATGACCACCAGGTAGGTGTATT. |
| | (SEQ ID NO. 11) |

Bioinformatics
Give each patient a donor number.
Maintain a record of the donor number and the patient that number represents internally within your office.
Complete the Case Report Form. Include the form with the donation.
Label Tubes and case report forms as follows: TranscriptPanel 002; Patient ID; Collection Date; Collection Time

Inclusion and Exclusion Methods As described for Example 1.

Analysis of the Study The gene-specific analysis will be considered successful of the levels of mRNA corresponding to the mutations coincide with the onset of clinical symptoms.

What is claimed is:

1. A method for conducting a small-scale clinical trial to assess the toxicity of an intervention comprising the steps of:
obtaining fewer than about fifty subjects, wherein one or more subjects will serve as the control group and at least twice as many subjects as in said control group will be subject to said intervention;
obtaining a sample of the subject's peripheral blood mononuclear cells (PBMC) at time of dosing (T);
dosing said subjects;
subsequently collecting further PBMC from said subjects (T+);
obtaining samples of total RNA from said collected PBMC;
analyzing said RNA for mRNA content;
amplifying said mRNA;
analyzing said mRNA for the ten mRNAs for which the quantity of expression is most changed when comparing the mRNA expressed in control subjects and the mRNA expressed in the intervention subjects;
identifying the genes that correlate with said ten mRNAs;
comparing the genetic expression in the control and intervention subjects;
optionally comparing the genetic expression of the control and intervention subjects with additional gene databases; and
assessing, based on the expression of said genes in the control and intervention group, the toxicity of said intervention.

2. The method of claim 1, wherein said subsequent collection of PBMC is at T+30 minutes, T+1 hour, T+2 hours, and T+4 hours.

3. The method of claim 1, wherein the expression of said genes is further analyzed to determine the metabolism of said intervention.

4. The method of claim 1, wherein the expression of said genes is further analyzed to determine the maximum effect of said intervention.

5. The method of claim 1, wherein the total number of said subjects is five.

6. A method for conducting a small-scale clinical trial to assess the toxicity of an intervention comprising the steps of:
obtaining fewer than about fifty subjects, wherein the subjects are divided into two roughly equal sized groups, each of which group will receive a different intervention;
obtaining a sample of each subject's peripheral blood mononuclear cells (PBMC) at time of dosing (T);
dosing said subjects according to their group;
subsequently collecting further PBMC from said subjects (T+);
obtaining samples of total RNA from said collected PBMC;
analyzing said RNA for mRNA content;
amplifying said mRNA;
analyzing said mRNA for the ten mRNAs for which the quantity of expression is most changed when comparing the mRNA expressed in each of the intervention groups;
identifying the genes that correlate with said ten mRNAs;
comparing the genetic expression in the control and intervention subjects;
optionally comparing the genetic expression of the control and intervention subjects with additional gene databases; and
assessing, based on the expression of said genes in the control and intervention group, which intervention causes the most significant result.

7. The method of claim 6, wherein said significant result is an indication of toxicity.

8. The method of claim 6, wherein said significant result relates to the comparative metabolism of the two interventions.

9. The method of claim 6, wherein said significant results relates to the maximum effect of the two interventions.

10. The method of claim 6, wherein said subsequent collection of PBMC is at T+30 minutes, T+1 hour, T+2 hours, and T+4 hours.

11. The method of claim 7, wherein the total number of said subjects is five.
